(19) United States
(12) Patent Application Publication
(10) Pub. No.: US 2021/0079358 A1
(43) Pub. Date: Mar. 18, 2021

(54) ASSEMBLOID - 3D MIMETIC TISSUE STRUCTURE BASED ON PATIENT- DERIVED MULTIPLE CELL TYPES AND METHOD OF MANUFACTURING THE SAME

(71) Applicant: POSTECH Research and Business Development Foundation, Pohang-si (KR)

(72) Inventors: Kunyoo SHIN, Pohang-si (KR); Eunjee KIM, Seoul (KR); Sungjune JUNG, Pohang-si (KR)

(21) Appl. No.: 17/005,674

(22) Filed: Aug. 28, 2020

(30) Foreign Application Priority Data
Sep. 16, 2019 (KR) 10-2019-0113709

(51) Int. Cl. C12N 5/071

(52) U.S. Cl. CPC C12N 5/0685; C12N 2513/00; C12N 2506/30; C12N 2502/1347; C12N 2502/02

(57) ABSTRACT

The present invention relates to a 3 dimensional mimetic tissue structure—“Assembloid” based on patient-derived multiple cell types to develop next generation organoid technology serving as a novel platform for new drug development and a disease model and a method of manufacturing the same, and more particularly, to a stem cell- or tumor cell-based 3D multicellular mimetic tissue structure manufactured by reconstituting epithelial or tumor cells with various cellular components of a microenvironment such as stromal cells, vascular cells, immune cells or muscle cells based on three-dimensional (3D) bioprinting, and a method of manufacturing the same. As the stem cell- or tumor cell-based 3D multicellular mimetic tissue structure containing the major factors of a tissue microenvironment, such as stromal cells, vascular cells, immune cells and muscle cells, designed according to the present invention is confirmed to mimic physiological and pathological characteristics of tissue in the body better than conventional organoids, normal and tumor assembloids may be used as a new platform for new drug development and a disease model. More specifically, together with 3D bioprinting technology, it is expected that in vitro bladder tissue and bladder tumor tissue are effectively used as a platform to develop precise and personalized therapeutic options for bladder related diseases including bladder cancer.

### CROSS-REFERENCE TO RELATED APPLICATION

This application claims priority to and the benefit of Korean Patent Application No. 10-2019-0113709, filed on Sep. 16, 2019, the disclosure of which is incorporated herein by reference in its entirety.

### BACKGROUND

### 1. Field of the Invention

The present invention relates to a 3 dimensional mimetic tissue structure (Assembloid) which is defined by ‘Organoids derived from tissue stem/tumor cells reconstituted with other components of the tissue stroma/microenvironment based on patient-derived multiple cell types and a method of manufacturing the same, and more particularly, to a multicellular mimetic tissue structure based on various normal cells, which is manufactured by constituting epithelial or tumor cells with various cellular components of a microenvironment such as stromal cells, vascular cells, immune cells or muscle cells based on three-dimensional (3D) bioprinting, and a method of manufacturing the same.

### 2. Discussion of Related Art

For in vitro modeling of a human disease, efforts have been made to develop organoids, which are three-dimensional organ structures exhibiting structures and functions similar to in vivo tissues. Unlike conventional two-dimensional (2D) monolayer culture tending to use cells from original tissues due to genetic mutation, organoids mimic primary tissue cells and have the capacity of self-renewal to construct cellular components structures of in vivo tissues. Currently, numerous tissues such as the intestines, brain, kidneys, lungs, pancreas, stomach, liver, prostate, and bladder are modeled in vitro.

Recently, 3D culture systems for pancreatic, prostate, colon, colorectal, breast, liver and bladder tumors have been constructed due to the development of patient-derived tumor organoids derived from tumors of cancer patients. These tumor organoids retain similar pathological characteristics to actual tumors, improving the possibility of developing novel therapeutic methods for cancer treatment.

Organoids are mainly derived from tissue-restricted adult stem cells and pluripotent stem cells (PSCs), and tumor organoids are derived from tumor cells. The generation of the organoids derived from these three types of cells has been used as a method of modeling key characteristics of organs and tissues to understand various aspects of human diseases including cancer. Although these organoids represent the potential for a variety of biological studies on normal tissues and cancer, questions remain because numerous factors including a microenvironment associated with a disease in vivo cannot be explained.

Although it is possible to overcome this limitation in the microenvironment associated with an in vivo disease by using PSC-derived organoids, these are usually immature or in an embryonic state, and thus require transplantation to exhibit the normal characteristics of adult tissue. Also, there is a problem in that PSC-derived organoids cannot be applied to a tumor model.

To overcome these problems, recent studies have reported the structures of organotypic organoids including various cell components, but these organotypic organoids still do not precisely mimic in vivo tissue because they use a traditional co-culture system or a simple mixture of cellular components without an organized structure, and moreover, there is a limitation in which most of the cellular components that constitute a tissue microenvironment or stroma are lacking.

Meanwhile, in a recent study, a method of manufacturing bladder organoids (Proc Natl Acad Sci USA. 2019 Feb. 20) is actively being studied, but there are no studies on a stem cell- or tumor cell-based multicellular mimetic tissue structure realizing a microenvironment present in normal tissue or tumor tissue and a method of manufacturing the same.

### SUMMARY OF THE INVENTION

Therefore, as a result of earnest research to construct an advanced mimetic tissue structure realizing a microenvironment present in normal tissue or tumor tissue in an organoid, the inventors reconstituted “Assembloid” by culturing organoids derived from stem cells or tumor cells along with the four major components of tissue stroma: stromal fibroblasts (cancer-associated fibroblasts in the case of tumor), endothelial cells, immune cells and muscle layers, first identifying a novel stem cell- or tumor cell-based 3D mimetic tissue structure, which includes organoids and microenvironment-constituting cells. Based on this, the present invention was completed.

Therefore, the present invention is directed to providing a method of manufacturing a 3D mimetic tissue structure (Assembloid), which includes the following steps:

(a) preparing an organoid;

(b) culturing the organoid in a medium containing fibroblasts and endothelial cells; and

(c) culturing the organoid cultured in step (b) in a medium containing muscle cells.

In addition, the present invention is directed to providing a mimetic tissue structure manufactured by the method.

However, technical problems to be solved in the present invention are not limited to the above-described problems, and other problems which are not described herein will be fully understood by those of ordinary skill in the art from the following descriptions.

To achieve the objects of the present invention, the present invention provides a method of manufacturing a 3D mimetic tissue structure, which includes the following steps:

(a) preparing an organoid;

(b) culturing the organoid in a medium containing fibroblasts and endothelial cells; and

(c) culturing the organoid cultured in step (b) in a medium containing muscle cells.

In one embodiment of the present invention, the organoid in step (a) may be manufactured by culturing any one of stem cells or tumor tissue.

In another embodiment of the present invention, the tumor tissue may be a bladder tumor tissue derived from a patient undergoing transurethral resection of bladder tumor (TURBT) or a cystectomy.

In still another embodiment of the present invention, the fibroblasts may be selected from the group consisting of mouse embryonic fibroblasts (MEFs) and cancer-associated fibroblasts (CAFs).

In yet another embodiment of the present invention, the muscle cells may be selected from the group consisting of primary bladder smooth muscle cells (BSMCs) and human smooth muscle cells (hSMCs).

In addition, the present invention provides a 3D mimetic tissue structure manufactured by the above method.

In one embodiment of the present invention, the tissue of the 3D mimetic tissue structure may be bladder tissue.

In another embodiment of the present invention, the tissue of the 3D mimetic tissue structure may be bladder tumor tissue.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A to 1E show that long-term cultured bladder organoids mimic the urothelium of native bladders: a schematic diagram illustrating experimental strategies for short-term and long-term culture of normal bladder organoids (FIG. 1A); short-term (18 days) subcultured organoids (base type (Ck5, green) and luminal type (Ck18, red); (FIG. 1B); bladder organoids at certain time points during long-term culture (FIG. 1C), results of comparing short-term (14 days) organoids with embryonic (E16) bladders (FIG. 1D), and results of comparing long-term (81 days) cultured organoids with adult (p8 week) bladders (Scale bars represent 100 μm (left) and 10 μm (right); FIG. 1E);

FIGS. 2A to 2J show that in vitro reconstituted three-layered miniature bladders show similar physiological activity to the tissue structure of in vivo bladders: an experimental scheme for reconstituting bladder organoids with stroma (FIG. 2A); a bladder organoid with stroma (FIG. 2B); a reconstituted bladder organoid with stroma (FIG. 2C); a quantification result of epithelial cell proliferation of a reconstituted bladder organoid with stroma treated with Vismodegib, SAG or DMSO for 7 days (FIG. 2D); a quantification result of stromal cell proliferation of a reconstituted bladder organoid with stroma treated with Vismodegib, SAG or DMSO for 7 days (FIG. 2E); an experimental scheme for generating three-layered miniature bladders (FIG. 2F); a three-layered miniature bladder (FIG. 2G); a wild-type bladder (FIG. 2H); a quantification result of epithelial cell proliferation of a three-layered miniature bladder treated with Vismodegib, SAG or DMSO for 7 days (FIG. 2I); and a quantification result of stromal cell proliferation of a three-layered miniature bladder treated with Vismodegib, SAG or DMSO for 7 days (FIG. 2J);

FIGS. 3A to 3I show that a miniature bladder mimics the physiological activity of an in vivo bladder and tissue dynamics during urinary tract infection (UTI): a schematic diagram for establishment of UTI models in vivo and in vitro (FIG. 3A); wild-type bladders 3 days after UPEC infection (FIG. 3B); miniature bladders 3 days after UPEC infection (FIG. 3C); a result of analyzing the expression of Gil1, Wnt2 and Wnt4 in epithelial cells or stroma of wild-type bladders through quantitative RT-PCR (FIG. 3D); a result of analyzing the expression of epithelial cells or stroma of a miniature bladder through quantitative RT-PCR (FIG. 3E); an experimental scheme for lineage tracing and clonal analysis of Ck5-expressing basal stem cells and their progeny to investigate clonal relationships during UTI-induced urothelial regeneration in vivo and in vitro (FIG. 3F); a result of analyzing bladders from TM-injected CK5^{CreERT2}; R26^{Rainbow/wt} mice before (UPEC day 0) and after (UPEC day 7) bacterial injury for four-color fluorescence (Scale bars represent 100 μm) (FIG. 3G); a result of analyzing miniature bladders derived from CK5^{CreERT2}; R26^{rainbow/wt} mice treated with 4-OHT before (UPEC day 0) and after (UPEC day 7) bacterial infection for four-color fluorescence (Scale bars represent 100 μm) (FIG. 3H); and a model with clone relationship during UTI-induced urothelial regeneration (FIG. 3I);

FIGS. 4A to 4E show the histopathology of human urothelial carcinoma confirmed by patient-derived, three-dimensionally reconstituted bladder tumor organoids: eight bladder tumor organoid lines established from patient-derived invasive urothelial carcinoma samples, analyzed by H&E staining and immunostaining for basal (Ck5, green) and luminal (Ck18, red) markers (FIG. 4A); the histopathology of a luminal P-1 tumor and a luminal P-6 tumor (FIGS. 4B and 4E); and the histopathology of a basal P-2 tumor and a basal P-3 tumor (FIGS. 4C and 4D);

FIGS. 5A to 5I are results of confirming in vivo tumor responses of reconstituted bladder tumor organoids to a stroma-mediated tumor, subtype-dependent anticancer agents and conventional chemotherapeutic drugs: results of analyzing luminal P-1 tumor subtypes (FIG. 5A) and luminal P-6 tumor subtypes (FIG. 5D) by immunostaining patient-derived reconstituted bladder tumor organoids including stromal cells treated with SAG, FK506 or a vehicle control for Ck18; results of analyzing basal P-2 tumor subtypes (FIG. 5B) and basal P-3 tumor subtypes (FIG. 5C) by immunostaining patient-derived reconstituted bladder tumor organoids including stromal cells treated with SAG, FK506 or a vehicle control for Ck5; a result confirmed from a dose-response curve for patient-derived bladder tumor organoids (red) and reconstituted tumor organoids with stromal cells (blue) derived from luminal P-1 tumors with respect to three chemotherapeutic drugs (cisplatin, gemcitabine and mitomycin C) (FIG. 5E); a result confirmed from a dose-response curve for patient-derived bladder tumor organoids (red) and reconstituted tumor organoids with stromal cells (blue) derived from basal P-2 tumors with respect to three chemotherapeutic drugs (cisplatin, gemcitabine and mitomycin C) (FIG. 5F); a result confirmed from a dose-response curve for patient-derived bladder tumor organoids (red) and reconstituted tumor organoids with stromal cells (blue) derived from basal P-3 tumors with respect to three chemotherapeutic drugs (cisplatin, gemcitabine and mitomycin C) (FIG. 5G); a result confirmed from a dose-response curve for patient-derived bladder tumor organoids (red) and reconstituted tumor organoids with stromal cells (blue) derived from luminal P-6 tumors with respect to three chemotherapeutic drugs (cisplatin, gemcitabine and mitomycin C) (FIG. 5H); and a heatmap of log IC₅₀ for three chemotherapeutic drugs used to treat four reconstituted patient-derived bladder tumor organoids with or without stroma (FIG. 5I);

FIGS. 6A to 6G are the result of confirming patient-derived urothelial carcinoma-like physiological activity of bladder tumor organoids with stroma produced by 3-bioprinting-based reconstitution: a schematic diagram of 3D bioprinting-based reconstitution process to generate bladder tumor organoids with stroma (FIG. 6A); a result of analyzing 3D bioprinted tumor organoids with stroma derived from a P-1 line through H&E staining and immunostaining for CK5 or CK18 (FIG. 6B); a result of analyzing 3D bioprinted tumor organoids with stroma derived from a P-3 line through H&E staining and immunostaining for CK5 or CK18 (FIG. 6C); a result of analyzing 3D bioprinted, patient-derived bladder tumor organoids with stroma treated with SAG, FK506 or vehicle control through immunostaining for Ck18 (luminal subtype) (FIG. 6D); a result of analyzing 3D bioprinted, patient-derived bladder tumor organoids with stroma treated with SAG, FK506 or vehicle control through immunostaining for Ck5 (basal subtype) (FIG. 6E); a 3D bioprinted, patient-derived luminal bladder tumor organoids with stromal cells treated with cisplatin and stained with caspase3, compared with those without stroma (FIG. 6F); and a patient-derived basal bladder tumor organoids with stromal cells treated with cisplatin and stained with caspase3, compared with those without stroma (FIG. 6G);

FIGS. 7A to 7D show whether reconstituted bladder tumor organoids having tumor microenvironments show tumor invasion and immune cell infiltration: an experimental scheme for generating bladder tumor organoids with stroma and an outer muscle layer (FIG. 7A); a result of analyzing bladder tumor organoids with stroma and an outer muscle layer, derived from a P-7 line (luminal, T1 stage) and a P-3 line (basal, T2 stage), through immunostaining for CK18 (luminal subtype) or CK5 (basal subtype) and α-SMA (smooth muscle layer) (FIG. 7B); an experimental scheme for generating bladder tumor organoids with stroma and tumor-reactive T cells (FIG. 7C); and bladder tumor organoids with stroma and reconstituted tumor-reactive T cells analyzed by bright field images and immunostaining for Ck5 (tumor cells), vimentin (stromal fibroblasts) and CD8 (CD8 T cells) (FIG. 7D);

FIGS. 8A to 8E show that single cell-derived bladder organoids are maintained for one year by short-term serial passaging or long-term culture: a result showing that single urothelial cells successfully generated bladder organoids over time (FIG. 8A); bright field images of short-term (9 days) subcultured organoids among bladder organoids from passage 1 to 20 (FIG. 8B); a quantification result for organoid-forming efficiency at each passage (FIG. 8C); a quantification result for the sizes of organoids cultured for 9 days at each passage (FIG. 8D); and an average size of long-term cultured organoids at the indicated day (FIG. 8E);

FIGS. 9A to 9E show that in vitro reconstituted, three-layered miniature bladders cultured in a spinning bioreactor recapitulate an in vivo tissue structure: representative images of a 3D printed, 12-well spinning bioreactor (FIG. 9A); bladder organoids analyzed by immunostaining for Ck5 and Ck18 to mark a basal layer and a luminal layer of the bladder epithelium (FIG. 9B); reconstituted bladder organoids with stroma analyzed by immunostaining for Ck5 and Ck18 to mark a basal layer and a luminal layer for the bladder epithelium (FIG. 9C); a reconstituted three-layered miniature bladder analyzed by immunostaining with Ck5, Ck18 and vimentin to mark basal layers and luminal layers of the bladder epithelium and stroma (FIG. 9D); and wild-type bladders analyzed by immunostaining with Ck5, Ck18 and vimentin for basal and luminal layers of the bladder epithelium and stroma (FIG. 9E);

FIGS. 10A to 10C show that in vitro reconstituted, three-layered miniature bladders mimic epithelial-stromal interaction of in vivo bladders: bladder organoids treated with Vismodegib, SAG or DMSO for 7 days, confirmed through immunostaining for Ck5 (urothelial) and vimentin (stroma) (FIG. 10A); reconstituted bladder organoids with stroma treated with Vismodegib, SAG or DMSO for 7 days, confirmed through immunostaining for Ck5 (urothelial) and vimentin (stroma) (FIG. 10B); and three-layered miniature bladders treated with Vismodegib, SAG or DMSO for 7 days, confirmed through immunostaining for Ck5 (urothelial) and vimentin (substrate) (FIG. 10C);

FIGS. 11A to 11C show the tissue dynamics of reconstituted miniature bladders derived from Rainbow mice during UTI: a schematic diagram of a Rainbow allele (FIG. 11A); single cell-labelled normal bladder (left panel) or bladder organoid (right panel) from a Rainbow mouse after TM treatment (FIG. 11B); and an experimental scheme for in vivo and in vitro lineage tracing of Ck5-expressing basal epithelial cells (FIG. 11C);

FIGS. 12A to 12I show the gene expression analysis of patient-derived tumor organoids for determining molecular subtypes of human invasive urothelial carcinoma, and relative expression levels of luminal markers (UPK1A, UPK2, ERBB2, FOXA1 and GATA3) and basal markers (UPK1A, UPK2, ERBB2, FOXA1 and GATA3) in eight patient-derived bladder tumor organoids, analyzed by quantitative RT-PCR: an RT-PCR analysis result for a P-1 line (FIG. 12A); an RT-PCR analysis result for a P-2 line (FIG. 12B); an RT-PCR analysis result for a P-3 line (FIG. 12C); an RT-PCR analysis result for a P-4 line (FIG. 12D); an RT-PCR analysis result for a P-5 line (FIG. 12E); an RT-PCR analysis result for a P-6 line (FIG. 12F); an RT-PCR analysis result for a P-7 line (FIG. 12G); an RT-PCR analysis result for a P-8 line (FIG. 12H); and molecular subtypes of eight patient-derived bladder tumor organoids, confirmed by gene expression analysis (FIG. 12I);

FIG. 13 is an experimental scheme for reconstituting patient-derived bladder tumor organoids with stroma;

FIG. 14 is an experimental scheme for testing responses of reconstituted bladder tumor organoids with stroma to various anticancer agents;

FIG. 15 shows that reconstituted bladder tumor organoids show in vivo tumor responses to stroma-mediated tumor and subtype-dependent anticancer agents;

FIG. 16 is an experimental scheme for 3D bioprinting-based reconstitution of patient-derived bladder tumor organoids with stroma;

FIG. 17 shows that 3D bioprinting-based, reconstituted bladder tumor organoids with stroma have physiological activity of patient-derived urothelial carcinoma;

FIGS. 18A and 18B show in vitro differentiation of human pluripotent stem cells into contractible smooth muscle cells: FIG. 18A is an experimental scheme for stepwise differentiation of hESCs into contractible hSMCs; and FIG. 18B shows hSMCs differentiated from hESCs on day 14, analyzed by immunostaining ((α-SMA (green) and vimentin (red));

FIGS. 19A to 19E show that reconstituted bladder tumor organoids with stroma, derived from BBN-induced murine urothelial carcinoma, mimic the tumor structure and pathophysiology of an endogenous BBN-induced bladder tumor: a schematic diagram for reconstituting mouse bladder tumor organoids with stroma (FIG. 19A); tumor organoids derived from a BBN-induced mouse bladder tumor, analyzed by H&E staining and immunostaining for Ck5, vimentin and CD31 (FIG. 19B); reconstituted bladder tumor organoids with stroma, analyzed by H&E staining and immunostaining for Ck5, vimentin and CD31 (FIG. 19C); an endogenous BBN-induced mouse bladder tumor, analyzed by H&E staining and immunostaining for Ck5, vimentin and CD31 (FIG. 19D); and reconstituted mouse bladder tissue organoids with stroma treated with SAG, FK506 or vesicle control, analyzed by immunostaining with CK5 (green) and vimentin (red) (FIG. 19E);

FIGS. 20A and 20B show the generation of tumor-reactive CD8 T cells by co-culturing tumor cells with isogenic lymphocytes: an experimental scheme for generating tumor-reactive T cells (FIG. 20A); and flow cytometry plots on tumor-reactive CD8 T cells expressing CD69 and IFNγ (FIG. 20B); and

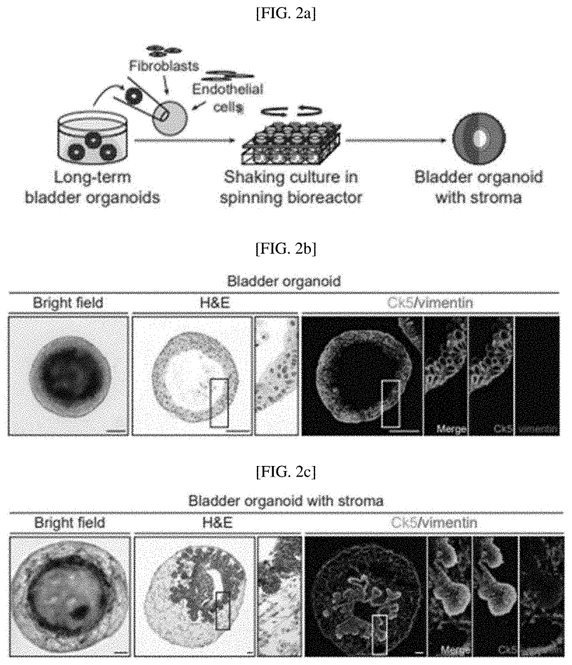

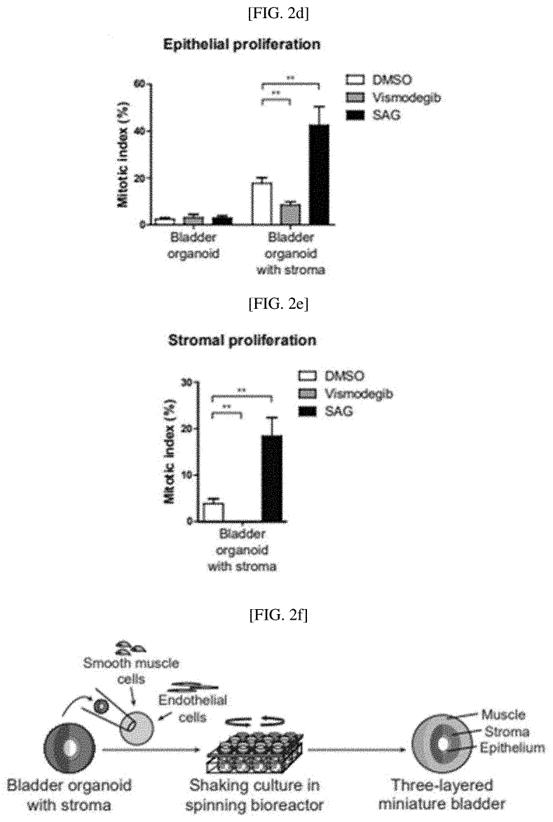

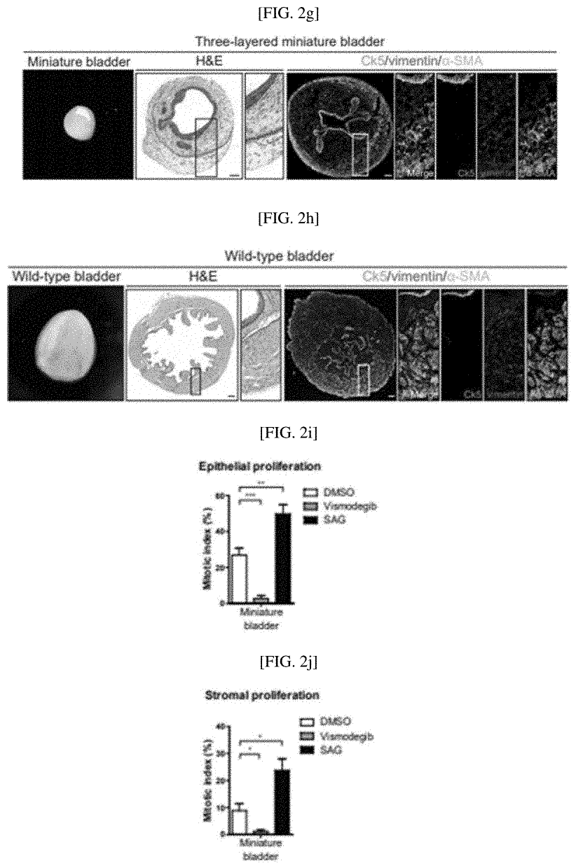

FIG. 2I illustrates a method of manufacturing a next generation mimetic tissue.

### DETAILED DESCRIPTION OF EXEMPLARY EMBODIMENTS

Hereinafter, the present invention will be described in detail.

As a result of earnest research to construct an advanced mimetic tissue structure realizing a microenvironment present in normal tissue or tumor tissue in an organoid, the inventors reconstituted “Assembloid” by culturing organoids derived from stem cells or tumor cells along with the four major components of tissue stroma: stromal fibroblasts (cancer-associated fibroblasts in the case of tumor), endothelial cells, immune cells and muscle layers, and allowed tissue reconstruction of muscle cells by culture with a single cell suspension obtained from smooth muscle cells and endothelial cells, first identifying a novel stem cell- or tumor cell-based multicellular mimetic tissue structure, which includes organoids and microenvironment-constituting cells. Based on this, the present invention was completed.

Therefore, the present invention relates to a method of manufacturing a 3D mimetic tissue structure (Assembloid), which includes the following steps:

(a) preparing an organoid;

(b) culturing the organoid in a medium containing fibroblasts and endothelial cells; and

(c) culturing the organoid cultured in step (b) in a medium containing muscle cells.

The term “organoid” used herein refers to a three-dimensional (3D) organ-like structure formed by culturing cells derived from tissue or embryonic stem cells. An organoid is a suffix that has the same meaning as an “organ” and also means “similar to an organ.” Organoids have a better arrangement of cells and their functions and have shapes and functions like functional organs due to a 3D culture method. Organoids have attracted attention with stem cell research and the development of 3D cell culture and research on optimizing growth and differentiation factors capable of differentiating into various tissues.

The term “3D mimetic tissue structure (Assembloids)” used herein is an advanced organoid containing various cell types such as epithelial or cancer cells and microenvironment tissue cells, and is used in the sense of higher-order organoids constituting in vitro 3D tissue derived from stem or tumor cells and the stroma—a tissue component inducing cell differentiation and expression of functions of the cells such as, and includes stromal cells, vascular cells, immune cells and muscle cells, but the present invention is not limited thereto.

In the present invention, the organoids in step (a) is preferably manufactured by culturing any one of stem cells or tumor tissue, and more particularly, a normal tissue organoid or tumor cell organoid is manufactured by culturing stem cells or tumor tissue.

In the present invention, the step cells used in step (a) may be selected from adult stem cells and pluripotent stem cells, and preferably, urothelial stem cells.

In the present invention, the tumor tissue used in step (a) may be tumor tissue derived from various tumors, preferably, bladder tumor tissue, and more preferably, bladder tumor tissue derived from a patient receiving transurethral resection of bladder tumor (TURBT) or a cystectomy.

In the present invention, the fibroblasts in step (b) may be cells constituting the major component of fibrous connective tissue, and preferably any one selected from the group consisting of mouse embryonic fibroblasts (MEFs) and cancer-associated fibroblasts (CAFs), but the present invention is not limited thereto.

In the present invention, the muscle cells in step (c) are preferably smooth muscle cells, which are muscle cells surrounding internal organs constituting tubes such as the stomach, digestive tract, blood vessels and bladder, and more preferably any one selected from the group consisting of primary bladder smooth muscle cells (BSMCs) and human smooth muscle cells (hSMCs), but the present invention is not limited thereto.

In addition, the present invention relates to a 3D mimetic tissue structure manufactured by the above-described method, and the tissue may be any one of intestinal tissue, intestinal tumor tissue, brain tissue, brain tumor tissue, kidney tissue, kidney tumor tissue, lung tissue, lung tumor tissue, pancreatic tissue, pancreatic tumor tissue, stomach tissue, stomach tumor tissue, liver tissue, liver tumor tissue, prostate tissue, prostate tumor tissue, bladder tissue, and bladder tumor tissue, and preferably bladder tissue or bladder tumor tissue.

In addition, the present invention provides a mouse bladder tumor mimetic tissue structure manufactured by culturing a mouse bladder tumor organoid differentiated from a BBN-induced bladder tumor in a medium containing mouse embryonic fibroblasts (MEFs), endothelial cells and tumor-reactive T cells, and a method of manufacturing the same.

The term “three-dimensional (3D) bioprinting” used herein is a technique also called cell printing or organ printing, and enables manufacture of computer-designed 3D constructs using various types of cells, biomaterials and biomolecules (Murphy, S V et al., Nature Biotechnology, 2014; 32: 773-785). This technique is attracting a great deal of attention in the field of tissue engineering for the purpose of artificial tissue or organ regeneration. Organs or tissues constituting the human body may be composed of biomaterials constituting various types of cells and the extracellular matrix. Research on regenerating required functional tissues through the manufacture of a cell construct similar to tissue constituting the human body using a 3D bioprinting technique is actively progressing. Recently, this technique has attracted a great deal of attention for developing an artificial bio-model for testing toxicity and efficacy of a new drug (Poliniab, A et al., Expert Opinion on Drug Discovery, 2014; 9(4): 335-352). In this printing process, a bio-ink is the most important key factor for precise patterning and securing a survival rate of cells in a bio printing process, and the characteristics of the ink are directly related to the process.

The inventors established the 3D mimetic tissue structure according to the present invention, manufactured by culturing normal bladder organoids and bladder tumor organoids and performing tissue reconstitution of cells constituting a microenvironment according to an embodiment, and a method of manufacturing the same.

In one embodiment of the present invention, a tissue structure similar to a reconstituted three-layered miniature bladder was confirmed in vitro, in vivo bladder-like physiological activity of the miniature bladder was confirmed, and it was confirmed that the pharmacological inhibition of a stromal Hedgehog (Hh) response using Vismodegib in the miniature bladder reduces the proliferation of epithelial cells and stromal cells by reconstituting a muscle layer of the miniature bladder, and upregulation of the stromal Hh activity using SAG increases the proliferation of epithelial cells and stromal cells, demonstrating that in vivo interactions between the epithelium and the stroma are repeated to induce cell proliferation by the three-layered miniature bladder (see Example 3).

In another embodiment of the present invention, to confirm the in vivo tumor response of a reconstituted bladder tumor organoid to a stroma-mediated, subtype-dependent anticancer agent, an in vivo tumor-like response of a reconstituted bladder tumor organoid to a stroma-mediated, subtype-dependent anticancer agent was confirmed by the stromal Hh activity effect of the bladder tumor organoid, and it was confirmed that in vivo responses of tumor organoids are precisely exhibited to various chemotherapeutic drugs due to a chemotherapeutic drug response of tumor stroma (see Example 6).

In still another embodiment of the present invention, tumor invasion and immune cell infiltration of reconstituted bladder tumor organoids having tumor microenvironments into a muscle layer were confirmed, thereby demonstrating that in vitro reconstituted organoids with stroma form platforms capable of modeling various biological aspects of tumors including muscular invasion and immune cell infiltration (see Example 8).

Hereinafter, to help in understanding the present invention, exemplary examples will be suggested. However, the following examples are merely provided to more easily understand the present invention, and not to limit the present invention.

### EXAMPLES

### Example 1. Preparation and Methods for Experiments

1-1. Mice

For lineage tracing experiments, CK5^{CreERT2} (JAX: 018394) mice were crossed with R26^{Rainbow/Rainbow} mice to obtain CK5^{CreERT2}; R26^{Rainbow/Rainbow} mice. Unless particularly stated, in all other experiments, C57BL/6 mice were used. In each experiment, mice were randomly selected from a cage for drug treatment. Procedures were performed under isoflurane anesthesia using a standard vaporizer. All procedures were performed according to the protocols approved by the Institutional Animal Care and Use Committee at POSTECH (IACUC number: POSTECH-2019-0055).

1-2. Human Bladder Tumor Samples

Human bladder tumor samples were obtained from the tissue bank of Seoul National University Hospital (SNUH). 0.5 to 1 cm³ specimens of bladder tumor tissue were acquired from patients undergoing TURB or a cystectomy according to a protocol approved by the SNUH Institutional Review Board (IRB). The tumor samples were transported to POSTECH after cryopreservation in 90% fetal bovine serum (FBS) containing 10% DMSO.

1-3. Culture of Normal Bladder Organoids

To isolate basal urothelial cells, mouse bladders were collected and inverted. The inverted bladders were incubated in DMEM supplemented with 10% FBS (Millipore) containing 500 U/mL collagenase/dispase (Sigma) at 37° C. for 2 hours, and the surface was scraped with a blade every 30 minutes. Isolated tissues were filtered through a 100 μm cell strainer (Falcon), a single-cell suspension was obtained after the lysis of red blood cells in ACK lysis buffer (Gibco), and cells were counted using a hemacytometer (Marienfeld). Urothelial cells were mixed with cold Matrigel (Growth Factor Reduced, Corning), layered onto a 24-well tissue culture dish (a 40 uL drop containing 6,000 cells) and incubated at 37° C. for 15 minutes. A pre-warmed organoid medium [advanced DMEM/F-12 (Gibco) supplemented with 10 mM HEPES (pH 7.4, Sigma), 10 mM nicotinamide (Sigma), 1 mM N-acetyl-L-cysteine (Sigma), GlutaMAX (Gibco), 1% penicillin/streptomycin (Gibco), 50 ng/mL mouse EGF (Peprotech), 0.5×B-27 (Gibco), 1 mM A8301 and 10 mM Y-27632] was added to make cells grown until analysis. In the case of subcultured organoids, bladder organoids in Matrigel were released by physical pipetting and collected in 15 mL tubes by centrifugation at 1,500 rpm and 4° C. for 5 minutes. Afterward, organoids were separated into single cells by incubation in 0.25% trypsin-EDTA (Welgene) containing Y-27632 at 37° C. for 10 minutes, followed by grinding for 1 to 2 minutes. Single cells were then seeded in Matrigel and cultured as described above. For Matrigel replacement, organoids in Matrigel were released by physical pipetting, collected in 15 mL tubes through centrifugation at 1500 rpm for 5 minutes at 4° C., mixed with fresh cold Matrigel and reseeded in the medium. The organoid medium was replaced with a fresh medium every 2 to 3 days, and Matrigel was replaced every 7 to 9 days.

1-4. Culture of Bladder Tumor Organoids

Tumor tissues obtained from patients were washed with DPBS, and then washed twice with DMEM supplemented with 10% FBS. The washed tumors were minced and incubated in DMEM containing 10% FBS and collagenase I and II (20 mg/mL each) at 37° C. for 1 hour and ground for 5 minutes every 30 minutes. The isolated tissues were spun down at 1,500 rpm for 5 minutes, resuspended in ACK lysis buffer, and then incubated for 5 minutes at room temperature. Dissociated clusters were washed with DMEM containing 10% FBS and filtered through a 100 μm cell strainer. The dissociated cell clusters were spun down, resuspended in Matrigel (Growth Factor reduced) and plated in the middle of one well of a 6-well plate. The plated drop was solidified in an incubator at 37° C. for 15 minutes, and a 2.5 mL organoid medium [advanced DMEM/F-12 supplemented with 10 mM HEPES (pH 7.4), 10 mM nicotinamide, 1 mM N-acetyl-L-cysteine, GlutaMAX, 1% penicillin/streptomycin, 50 ng/mL mouse EGF, 0.5×B-27, 1 mM A8301 and 10 mM Y-27632] was added to the well. The medium was replaced once every 2 to 3 days. For passaging, 1 mg/mL collagenase/dispase was added to the medium, followed by incubation for 1 hour at 37° C. to digest the Matrigel. Subsequently, organoids were centrifuged at 1,500 rpm for 3 minutes, washed with PBS, and then spun down. Then, 0.05% trypsin-EDTA (Welgene) was added, organoids were incubated at 37° C. for 5 minutes, followed by mechanical grinding into small cells by pipetting. The organoids were passaged in a 1:3 or 1:4 ratio every 1 to 2 weeks.

For the culture of mouse bladder tumor organoids, BBN-induced bladder tumors were ground and then incubated in DMEM containing collagenase I and II (20 mg/mL each) and thermolysin (250 KU/mL, Millipore) at 37° C. for 2 hours, and ground for 5 minutes every 30 minutes. A single-cell suspension was obtained through filtration through a 100 μm cell strainer. After the lysis of red blood cells in ACK lysis buffer, the cells were washed with DMEM containing 10% FBS and counted using a hemacytometer (Marienfeld). Single tumor cells were embedded in Matrigel, and incubated in an organoid culture medium [advanced DMEM/F-12 supplemented with 10 mM HEPES (pH 7.4), 10 mM nicotinamide, 1 mM N-acetyl-L-cysteine, GlutaMAX, 1% penicillin/streptomycin, 50 ng/mL mouse EGF, 0.5×B-27, 1 mM A8301 and 10 mM Y-27632]. To generate stocks, dissociated organoids were frozen in 90% FBS/10% DMSO, and stored in liquid nitrogen. Cryopreserved stocks were successfully recovered after freezing. It was confirmed that 40 to 50% of the cell clusters form organoids.

1-5. Reconstitution of Three-Layered Miniature Bladders

To generate normal bladder organoids with stroma, normal bladder organoids cultured for a long time (>6 months) were mixed with mouse embryonic fibroblasts (MEFs) and endothelial cells (HULEC, ATCC) at 3.75×10⁴ cells/μL and 5×10³ cells/μL, respectively, along with 3 to 4 μL of Matrigel. Before additional analysis, these bladder organoids with stroma were cultured in an organoid medium in a spinning bioreactor for 7 days. The 12-well version of the spinning bioreactor was manufactured by 3D printing according to the method described in Qian, X. et al. Brain-Region-Specific Organoids Using Mini-bioreactors for Modeling ZIKV Exposure. Cell 165, 1238-1254, doi:10.1016/j.cell. 2016.04.032 (2016). In brief, individual components such as a plate cover (Project MJP 2500, 3D Systems, USA), a rotating shaft (Single Plus, Cubicon, Korea), a fixed rod (Single Plus, Cubicon, Korea) and a gear (Single Plus, Cubicon, Korea) were printed using a 3D printer in a stackable version, and the components were assembled to manufacture a spinning bioreactor. To obtain an outer muscle layer for the development of three-layered miniature bladders, primary bladder smooth muscle cells (BSMCs) were isolated from mice. For isolation, mouse bladders were collected and inverted, and after a urothelial surface was removed by scraping with a blade, outer muscle walls were incubated in DMEM with 10% FBS containing collagenase I and II (2 mg/mL each) for 1 hour at 37° C. and ground every 15 minutes. A dissociated BSMC suspension by additional culture in DMEM with 10% FBS was filtered through a 100 μm cell strainer and centrifuged, thereby obtaining a single-cell suspension. To generate three-layered miniature bladders, each bladder organoid with stroma cultured for 7 days was put into 3 to 4 μL of a Matrigel medium containing BSMCs and HULECs at 3×10⁴ cells/μL and 3×10³ cells/μL, respectively. The miniature bladders generated as described above were cultured in a spinning bioreactor for 3 to 4 days, and then used for the experiments. To generate Rainbow miniature bladders, the bladder tissues derived from CK5^{CreERT2}; R26^{Rainbow/Rainbow} mice were cultured for a long time, and reconstituted with MEFs and HULECs. The reconstituted organoids were reconstituted with BSMCs derived from CK5^{CreERT2}; R26^{Rainbow/Rainbow} mice.

1-6. In Vivo and In Vitro UTI Models

For UTI models, UT189, which is a uropathogenic strain of E. coli (UPEC) isolated from human patients with UTI, was cultured for 4 hours with shaking, and a bacterial pellet was generated by centrifugation at 4,200 rpm for 8 minutes at room temperature. The pellet was washed twice with 5 mL PBS, centrifuged and resuspended in 1 mL physiological saline. To produce an in vivo UTI mouse model, anesthetized female mice between the age of 6 to 9 weeks were subjected to transurethral injection of UPEC at a concentration of 2×10¹⁰ CFU/mL in 50 μL. All transurethral injection procedures were performed under isoflurane anesthesia with a standard vaporizer. To generate an in vitro UTI mouse model, the organoid culture medium of the miniature bladder was replaced with an antibiotic-free fresh medium one day before microinjection. A bacterial culture was prepared as described above, and UPEC was microinjected directly into the lumen of miniature bladders using a microinjector (FemotoJet4i; Eppendorf) at an injection pressure of 40 hPa, an injection time of 0.5 s and a compensation pressure of 11 hPa. The injected miniature bladders were maintained with shaking and analyzed at indicated time points.

1-7. Lineage Marking and Tracing Experiments

To permanently label Ck5-expressing cells in vivo, 8 mg TM (based on 30 g of body weight) was orally administered to CK5^{CreERT2}; R26^{Rainbow/Rainbow} female mice three times a day. Five days after the last TM administration, UTI189 was transurethrally injected into the mice. At the indicated time points after injection, mice were sacrificed and bladders were collected. The collected bladders were fixed in 10% neutral-buffered formalin for 6 hours, washed with PBS three times, incubated overnight in 30% sucrose, and embedded in an OCT compound (Tissue-Tek). Tissue sections were mounted with a Prolong Gold mounting reagent (Invitrogen), and analyzed with four-color fluorescence. For in vitro lineage tracing, miniature bladders derived from CK5^{CreERT2}; R26^{Rainbow/Rainbow} mice were treated with 2 μM 4-hydroxytamoxifen (4-OHT) for 2 days to permanently label Ck5-expressing cells. After 4-OHT treatment, UT189 was microinjected into miniature bladders, and then the miniature bladders were collected at the indicated time points. The collected miniature bladders were fixed in 10% neutral-buffered formalin for 4 hours, washed with PBS for three times, incubated in 30% sucrose overnight, and embedded in an OCT compound. Sections were mounted with a Prolong Gold mounting reagent and analyzed with four-color fluorescence. Four-color fluorescence images were analyzed by confocal microscopy (Leica SP5 or Olympus FV1000).

1-8. Reconstitution of Bladder Tumor Organoids with Stroma

To generate patient-derived tumor organoids with stroma, human tumor organoids were cultured for 10 days, and mixed with stromal components containing patient-derived CAFs and HULECs at concentrations of 5×10⁴ cells/μL and 4×10³ cells/μL, respectively, in Matrigel. And then, 5 μL drops of tumor organoids with stroma were cultured in an organoid medium in a spinning bioreactor for 7 days. To reconstitute patient-derived bladder tumor organoids with stroma and an outer muscle layer, the drops of tumor organoids with stroma were added to 5 μL Matrigel containing human smooth muscle cells at a concentration of 5×10⁴ cells/μL. The generated tumor organoids were cultured in an organoid medium in a spinning bioreactor until analysis.

To generate mouse bladder tumor organoids with stroma, mouse bladder tumor organoids derived from BBN-induced bladder tumors were cultured for 10 days, and mixed with stromal components containing MEFs and HULECs at concentrations of 5×10⁴ cells/μL and 4×10³ cells/μL, respectively. Subsequently, 5 μL drops of tumor organoids with stroma were cultured in an organoid medium in a spinning bioreactor for 7 days.

To generate mouse bladder tumor organoids containing stroma and tumor-reactive T cells, mouse bladder tumor tissues were cultured for 10 days, and mixed with a stromal component containing MEFs, HULECs and tumor-reactive T cells at 5×10⁴ cells/μL, 4×10³ cells/μL and 2×10⁴ cells/μL, respectively. Subsequently, 5 μL drops of tumor organoids with stroma and tumor-reactive T cells were cultured in an organoid medium supplemented with 50 ng/mL IL-2 (Peprotech) in a spinning bioreactor for 7 days.

1-9. Drug Treatment and Response of Bladder Organoids

Normal bladder organoids were treated with 50 μM Vismodegib (Abmole), 100 nM SAG (Millipore) or DMSO and cultured in a spinning bioreactor for 7 days. Bladder tumor organoids were treated with 300 nM SAG, 5 μM FK506 (Cayman Chemical) or DMSO, and cultured in a spinning bioreactor for 7 days. Human bladder tumor organoids were cultured in an organoid medium for 7 days in a spinning bioreactor before treatment with chemotherapeutic drugs. Subsequently, tumor organoids were cultured for 48 hours in the presence of cisplatin (0, 0.2, 0.5, 1, 2, 5, 10, 50 and 100 μm, Sigma), gemcitabine (0, 0.2, 0.5, 1, 2, 5, 10, 50 and 100 μM, Sigma) or mitomycin C (0, 0.2, 0.5, 1, 2, 5, 10, 50 and 100 μM, Sigma). To produce dose-response curves for the three chemotherapeutic drugs, cell viability was assayed using a CellEvent™ Caspase-3/7 Green Detection Reagent (Thermo Fisher) according to the manufacturer's instructions. Forty-eight hours after treatment with cell therapeutic agent, bladder tumor organoids were fixed in 4% PFA at 4° C. for 15 minutes, washed with PBS three times, cultured in 30% sucrose overnight, and embedded in an OCT compound. Capase-3/7-stained cells in the bladder tumor organoids were counted based on green fluorescence signals, and dose-response curves were generated using GraphPad Prism ver.6.

1-10. Differentiation of Human Smooth Muscle Cells

H9 human embryonic stem cells (hESCs) were cultured to 30% confluence in an mTeSR (STEMCELL) medium on a Matrigel-coated plate with daily medium replacement. Mesodermal lineage differentiation was initiated by culturing the cells with DMEM/F12 (Gibco) supplemented with knockout Serum Replacement (Invitrogen), 1×MEM non-essential amino acids (Gibco), β-mercaptoethanol (Gibco), 1×Glutamax, 1% penicillin/streptomycin, 10 μM Y-27632, 10 ng/mL activin A (Peprotech) and 20 ng/mL BMP4 (Peprotech). Afterward, human smooth muscle cells (hSMCs) were differentiated by culturing the cells in DMEM/F12 containing 5% FBS, 1% penicillin/streptomycin, 5 ng/mL PDGF-BB (Peprotech) and 2.5 ng/mL TGFβ (Peprotech) for 3 to 14 days. The medium was replaced every 2 days.

1-11. Reconstitution of Bladder Tumor Organoids by 3D Bioprinting

Tumor cells derived from patient-derived bladder tumor organoids were embedded in 40 μL Matrigel at a density of 5×10⁵ cells/mL, and cultured in an organoid culture medium for 10 days. To prepare a bio-ink mixture for the reconstitution of tumor organoids with stroma by 3D bioprinting, 6 wells containing the embedded tumor organoids were treated with 0.5 mg/mL of a collagenase/dispase solution at 37° C. for 30 minutes, washed and centrifuged. The collected organoids were resuspended in 1 mL Matrigel (Growth Factor Reduced) containing patient-derived CAFs and HULECs at 4×10⁷ cells/mL and 4×10⁶ cells/mL, respectively. The reconstituted bio-ink was put into a sterilized 10 mL syringe with a 20-gauge needle, and stored on ice for less than 5 minutes before printing. A jet-type 3D printer (in vivo, Rokit, Korea) was used to reconstitute tumor organoids with stroma as described above. A G-code based script was written by Creator K software (Rokit, Korea) to print a 5×6 array of 10 μL drops. Each drop was dispensed on a parafilm at 8-mm intervals by extrusion. The temperature of a syringe insulator was set to 4° C. throughout the process. To avoid sedimentation of cells in the bio-ink mixture, each array was printed within 3 minutes. After dispensing, drops were inverted and crosslinked in an incubator (37° C., 5% CO₂) for 15 minutes. To exclude unstable products, the first two drops were discarded, and the remaining product was separately transferred to a 12-well spinning bioreactor containing a 2 mL organoid culture medium. The culture plate was incubated at 37° C., and the culture medium was replaced every 2 days until analysis.

1-12. Generation of Tumor-Reactive T Cells

Total lymph nodes and spleens were isolated from C57/BL6 mice. Tissues were ground with 3 mL PBS and filtered through a 100 μm cell strainer. Cell suspensions were collected in a 15 mL tube, and cell pellets were generated by centrifugation at 1,500 rpm and 4° C. Red blood cells in the lymphocyte pellets were removed with ACK lysis buffer. The isolated lymphocytes were cultured overnight with 10% FBS-containing RPMI (Welgene) supplemented with 5 ng/mL IL-2 in a 96-well tissue culture plate pre-coated with 0.5 μg/mL anti-CD3 (BD) and 0.5 μg/mL anti-CD28 (BD) at 37° C. To prepare the tumor cells for stimulating lymphocytes, BBN-induced mouse bladder tumor organoids from isogenic C57BL/6 mice were cultured for 7 days and treated with 200 ng/mL IFNγ (Peprotech) overnight at 37° C. The next day, the stimulated tumor organoids were separated into single cells and resuspended in a lymphocyte medium. The dissociated tumor cells and lymphocytes were co-cultured at a 1:20 ratio for 2 weeks in the presence of anti-CD28, IL-2 and 20 μg/mL anti-PD-1 (Bio X Cell) antibodies.

1-13. Flow Cytometry Analysis

The lymphocytes co-cultured with the tumor cells were collected, and re-stimulated with a cell stimulation cocktail for 4 hours in the presence of a protein transport inhibitor (eBioscience). Afterward, the cells were washed twice with FACS, and a surface marker was stained for 30 minutes at 4° C. For intracellular staining, a single-cell suspension was fixed, and permeability was maintained with an eBioscience staining buffer set. The following antibodies were used: anti-CD4-BUV395 (BD, GK1.5), anti-Zombie-Aqua (BioLegend), anti-CD8a-BV650 (BD, 53-6.7), anti-CD45.2-BV605 (BD, 104), anti-B220-BV710 (BD, RA3-6B2), anti-CD11b-PerCP-Cy5.5 (BD, M1/70), anti-TCRβ-APC-Cy7 (BD, H57-597), anti-CD69-PacificBlue (BioLegend, H1.2F3) and anti-IFNγ-PE (eBioscience, XMG1.2). The cells were analyzed on LSR II (BD) and data was processed with FlowJo software (Tree Star).

1-14. Quantitative RT-PCR

Tissues were homogenized by grinding and trypsin treatment, and RNA was extracted using an RNeasy Plus Mini Kit (Qiagen). The RNA samples were dissolved in RNase-free water, and then their concentrations and purities were measured using a spectrophotometer. For quantitative RT-PCR of mRNA transcripts, primary cDNA was synthesized using a High-Capacity cDNA Reverse Transcriptase Kit (Applied Biosystems) containing oligo DT. Quantitative RT-PCR was performed using SYBR Green Supermix (Applied Biosystems) and a one-step cycler (Applied Biosystems). Gene expression was normalized to a housekeeping gene HPRT.

1-15. Histological Analysis

Tissue specimens were pre-fixed in 10% neutral-buffered formalin for 24 hours and embedded in paraffin. To form agarose blocks, organoids were embedded in a 3% agarose gel, fixed in 10% neutral-buffered formalin for 24 hours and embedded in paraffin. The paraffin block was cut into 4-μm sections using a microtome. Slides were stained with hematoxylin and then counter-stained with eosin for histological analysis.

1-16 Immunohistochemistry

Tissues and organoids were fixed in 4% PFA, embedded in an OCT compound, and cut into 10 to 25-μm sections using a Microm cryostat (Leica). For immunohistochemistry, frozen sections were post-fixed in 4% PFA at 4° C. for 20 minutes. The sections were washed with PBS three times, blocked with 2% goat serum and PBS containing 0.25% Triton X-100 (PBS-T) for 1 hour, and incubated with primary antibodies diluted in blocking buffer in a humidified chamber overnight at 4° C. Primary antibodies diluted in blocking buffer are as follows: rat anti-Ck18 (1:500, DSHB); rabbit anti-Ck5 (1:500, Abcam); chicken anti-vimentin (1:500, Millipore); mouse anti-uroplakin 3 (1:50, Fitzgerald); mouse anti-alpha smooth muscle actin (1:200, Abcam); hamster anti-CD31 (1:200, Abcam), rabbit anti-Ki67 (1:500, Abcam) and rabbit anti-caspase 3 (1:200, Cell Signaling). Afterward, the sections were washed three times with 0.25% PBS-T, and incubated with Alexa Fluor 488, 594, 633 or 647-conjugated secondary antibodies, which were suitably diluted 1:1,000, in 0.25% PBS-T, along with DAPI, at room temperature for 1 hour. The conjugated antibodies, anti-CD8a-BV510 (1:200, BD), were incubated in 0.25% PBS-T along with DAPI at room temperature for 1 hour. The sections were washed twice with 0.25% PBS-T, and mounted on slides with a Prolog Gold mounting reagent.

1-17. Data Analysis

Statistical analysis was performed using GraphPad Prism ver.6. All data is expressed as +/−s.e.m. The comparison between groups was performed by a two-tailed Student's test. P<0.05 was considered statistically significant.

### Example 2. Confirmation of Mimetic Property of the Urothelium of Native Bladder by Long-Term Cultured Bladder Organoid

2-1. Confirmation of Mature Bladder Tissue Differentiation of Urothelial Stem Cell

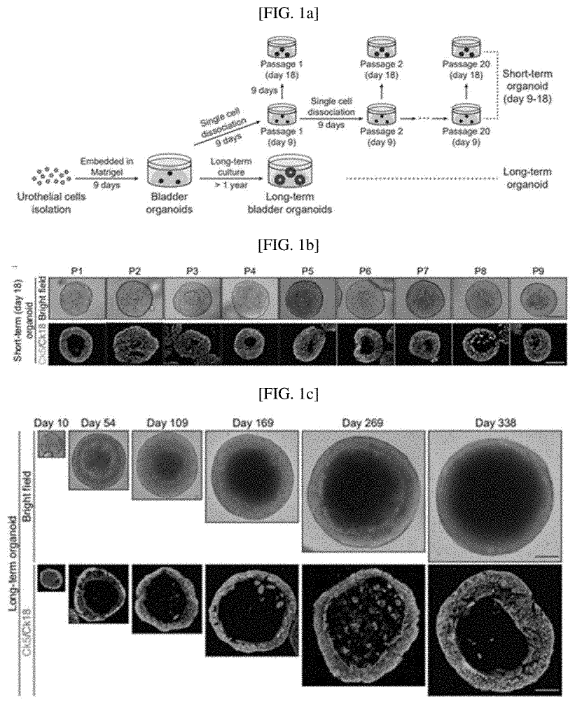

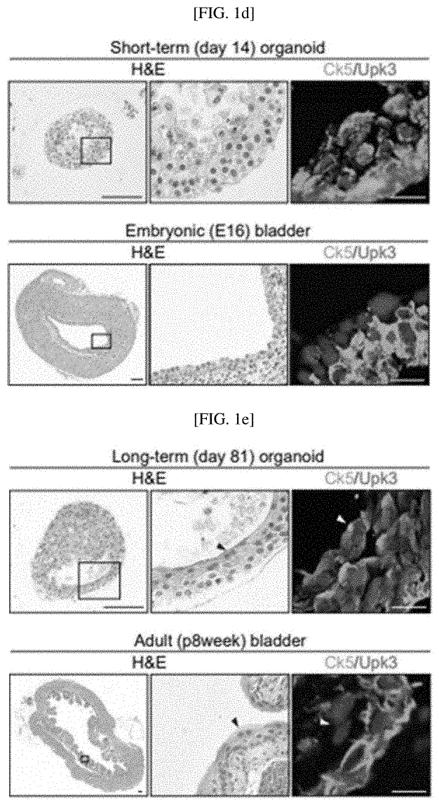

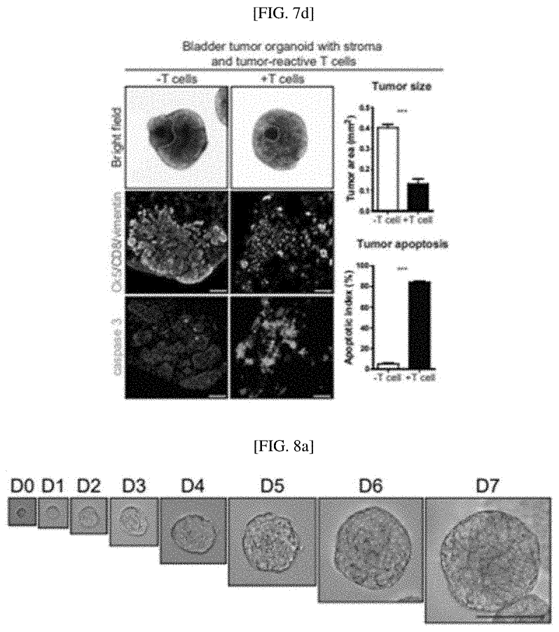

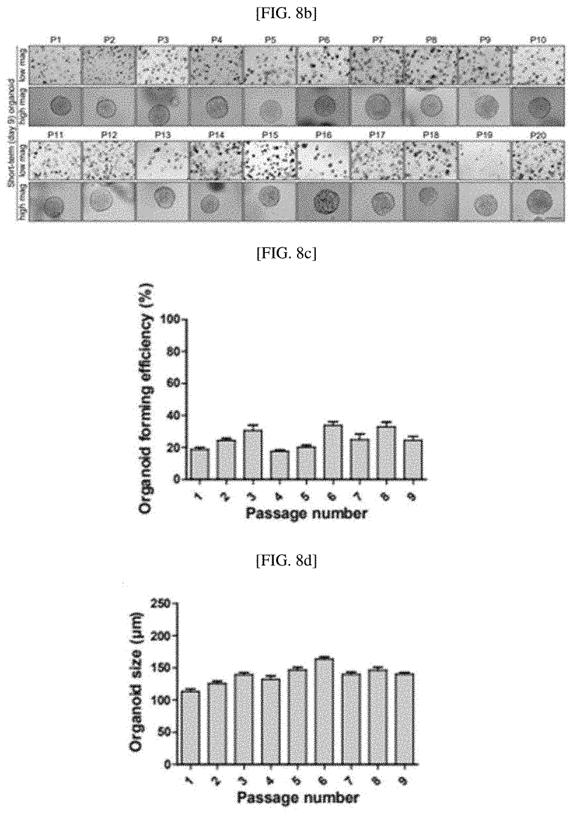

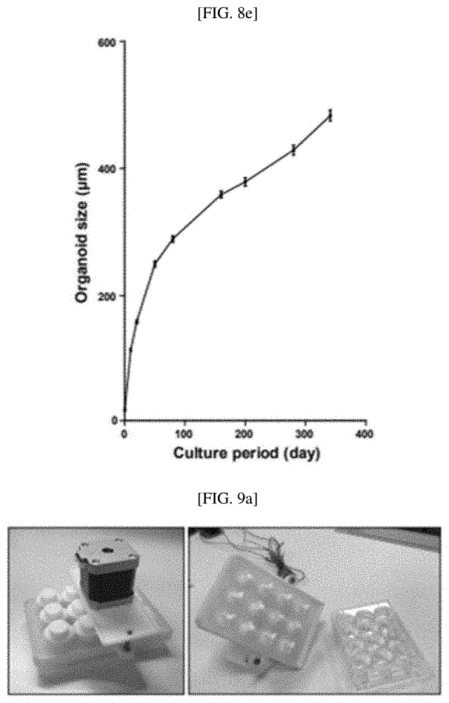

To confirm urothelial stem cells capable of forming a bladder tissue structure in vitro, and confirm whether organoids generated by the stem cells develop into mature bladder tissue including multiple layers of similarly differentiated epithelial cells as mature bladder tissue in vivo, as shown in FIG. 1A, two culture systems maintaining bladder organoids for a long time were established. For long-term serial passaging of short-term cultured organoids, as shown in FIG. 8A, primary bladder organoids generated by culturing single urothelial stem cells cultured in a 3D environment for 7 to 9 days were dissociated into single cells, and subcultured to form new bladder organoids. This process was repeated twenty times for 6 months, and as shown in FIG. 8B, it was confirmed that bladder tissue was successfully formed from each bladder. Therefore, it was confirmed that bladder organoids were generated from urothelial stem cells having self-regenerating ability. In addition, as shown in FIGS. 8C and 8D, it was confirmed that an average size of approximately 30% of urothelial cells in the bladder organoid cultured for 9 days is 140 μm, and organoid forming efficiency and an organoid size are maintained consistently.

In addition, the bladder organoids cultured for 9 days were further cultured for 9 days, and the bladder organoids cultured for 18 days were analyzed by immunostaining for the basal epithelial marker Ck5 and the luminal epithelial marker Ck18.

As a result, as shown in FIG. 1B, the bladder organoid cultured for 18 days consists of an outer basal epithelial layer expressing Ck5 and an inner luminal layer having the central lumen expressing Ck18, demonstrating that it mimics the multi-layered epithelium.

2-2. Confirmation of Long-Term Growth of Bladder Cell Membrane Organoid without Serial Passaging

As shown in FIG. 1C, it was confirmed that bladder organoids derived from urothelial stem cells were successfully cultured, and grown in vitro for over 1 year. In addition, it was confirmed that, as the bladder organoids mature, the central lumen is formed and more differentiated from Ck18+ layer intermediate cells and Ck5+ basal cells below the Ck18+ layer, and as shown in FIG. 8E, it was confirmed that the multilayered urothelial organoids cultured for a long time gradually increase and grow to a size of 500 μm after one year of culture.

In addition, as shown in FIGS. 1D and 1E, it was confirmed that uroplakin 3 (Upk3), which is the marker for terminally-differentiated, multinucleated umbrella cells that appear only in the adult bladder, is expressed in the luminal layer of the long-term cultured bladder organoids (81 days), but is not shown in the short-term cultured bladder organoids (14 days) similar to the early bladder.

From this result, it was confirmed that the long-term cultured bladder organoids recapitulate the normal bladder development from an embryonic state to an adult state.

In addition, it was confirmed that the long-term cultured bladder organoids represent the mature urothelial cells of the bladder having multiple layers of epithelial cells as well as Upk+ and Ck18 differentiated from luminal cells in an inner layer that is the line of a luminal space and an outer layer that contains a Ck5+ basal layer.

From the result of Example 2, a long-term culture system of normal bladder organoids that continuously expand normal bladders, self-organize a urothelial structure, and mimic mature urothelial cells was confirmed.

### Example 3. Confirmation of In Vivo Bladder-Like Tissue Structure and Physiological Activity of In Vitro Reconstituted Three-Layered Miniature Bladder

3-1. Confirmation of In Vivo Bladder-Like Tissue Structure of In Vitro Reconstituted Three-Layered Miniature Bladder

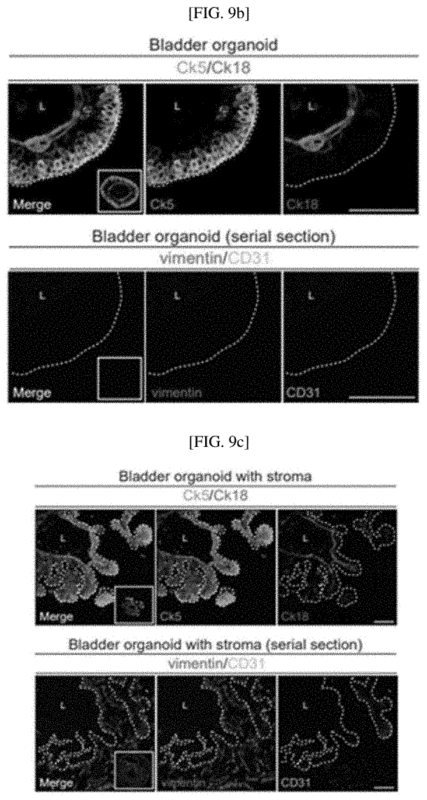

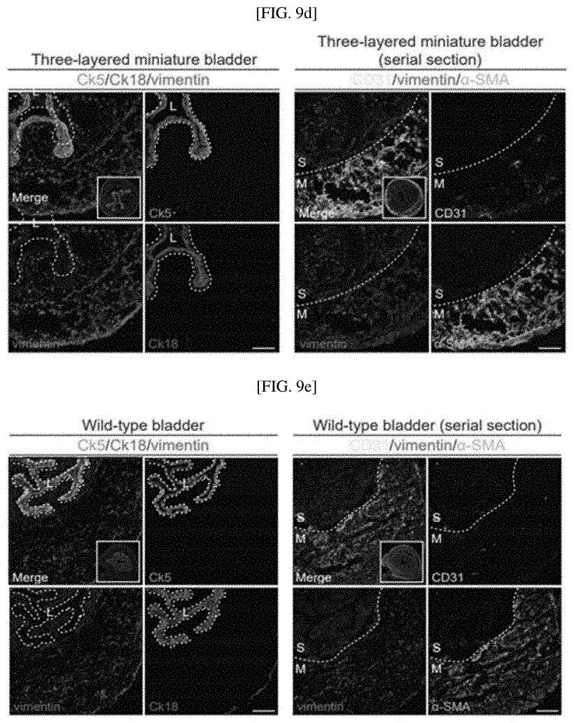

The inventors intended to determine that tissue stroma is a critical tissue component serving as a gap between stem cells for stimulating cell proliferation and differentiation and providing structural support, and to develop bladder organoids including tissue stroma. As shown in FIG. 2A, to mimic urothelial cells including mature stroma, bladder organoids cultured for a long period of over 200 days were reconstituted with two components of tissue stroma, such as fibroblasts and endothelial cells. In addition, as shown in FIG. 9A, to promote the growth and organization of bladder organoids with stroma, reconstituted organoids were cultured in a spinning bioreactor developed using 3D printing.

The cultured organoids were analyzed by immunohistochemical analysis, and as shown in FIGS. 2C and 9C, it was confirmed that, compared with conventional long-term bladder organoids only mimicking the urothelial portion of the bladder, the reconstituted organoids include a Ck5+ urothelial layer surrounded by a thick layer in which stromal components consisting of stromal fibroblasts are marked by the expression of vimentin and endothelial cells are marked by the expression of CD31.

In addition, similar to the reconstituted normal urothelial cells and the long-term urothelial organoids (FIG. 9B), it was confirmed that epithelial portions of the reconstituted organoids with stroma have a tissue layer structure consisting of a Ck5+ basal layer and a Ck18+ luminal layer (FIG. 9C).

From this result, it was confirmed that bladder organoids showed smooth and flat Ck5+ epithelial layers (FIG. 2B), the reconstituted bladder organoids with stroma include rough Ck5+ epithelial layers having several folds, which is one of the important characteristics of bladder urothelial cells in vivo (FIG. 2C).

3-2. Confirmation of In Vivo Bladder-Like Physiological Activity of In Vitro Reconstituted Three-Layered Miniature Bladder

Cell proliferation in the bladder requires a stromal Hedgehog (Hh) response, and is mediated by Hh/Wnt signaling feedback between the epithelium and stroma. To confirm whether the reconstituted bladder organoids showed stroma-mediated cell proliferation as the in vivo bladder, reconstituted bladder organoids were treated with Vismodegib or SAG, which pharmacologically inhibit the reconstituted bladder organics, was treated to activate the substrate Hh reaction

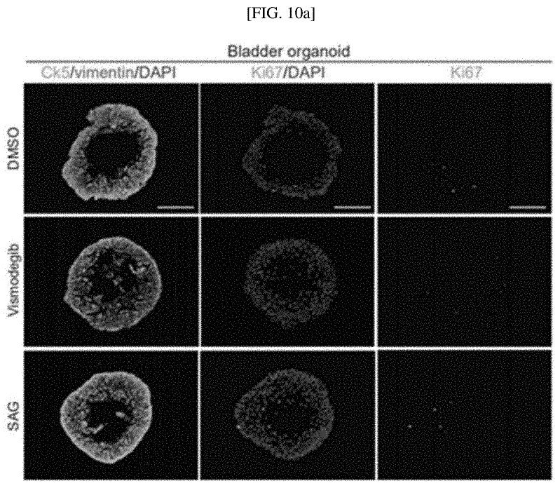

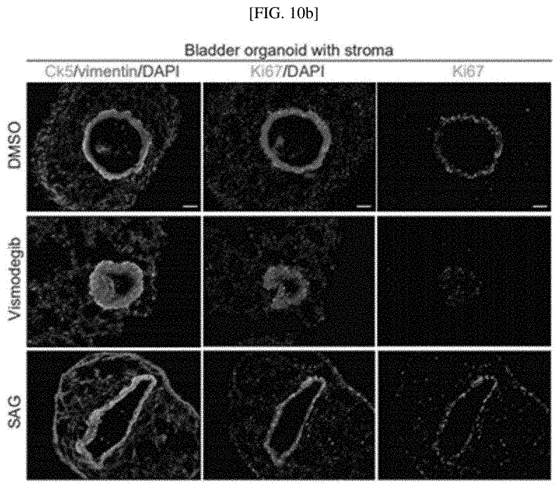

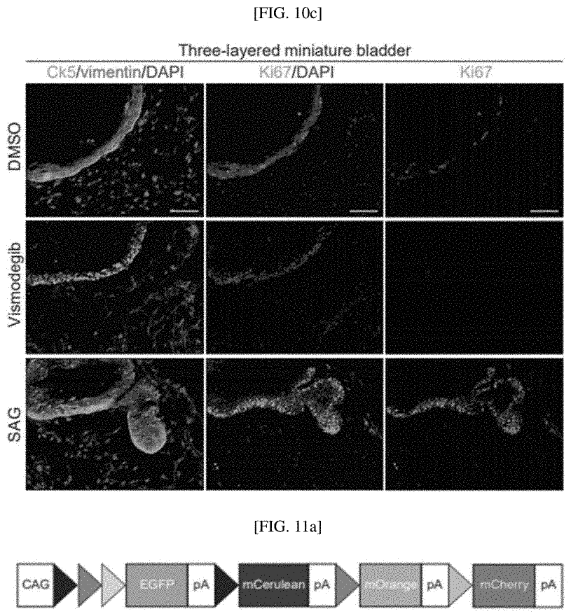

As a result, as shown in FIGS. 2D and 10A, it was confirmed that, due to the induction of the Hh response occurring only in the presence of tissue stroma, urothelial organoids without stroma does not respond to an Hh antagonist or agonist, and as shown in FIGS. 2D, 2E and 10B, reconstituted bladder organoids with stroma bring about a significant decrease in the proliferation of epithelial cells and stromal cells by Vismodegib treatment and a significant increase in the proliferation of epithelial cells and stromal cells by SAG treatment, confirming the pharmacological manipulation of Hh pathway activity.

From this result, it was confirmed that the reconstituted bladder organoids physiologically recapitulate the interaction between the epithelium and stroma of the normal bladder by maintaining functional stroma required for reciprocal Hh/Wnt signaling feedback.

3-3. Development of Muscle Layer of In Vitro Reconstituted Three-Layered Miniature Bladder

To further develop miniature bladders that precisely mimic the tissue structure of the adult bladder with the tissue structure of an outer muscle layer, as shown in FIG. 2F, a reconstitution procedure for generating bladder tissue with stroma and a muscle layer is performed, and immunohistochemical analysis was performed to confirm whether in vitro three-layered miniature bladders structurally represent the tissue organization of in vivo adult bladders.

As a result, as shown in FIGS. 2G and 2H, it was confirmed that the three-layered miniature bladder consists of three distinct compartments that are tightly organized to form a bladder-like structure. In addition, as shown in FIG. 9D, it was confirmed that a three-layered miniature bladder includes a central lumen, and a multilayered epithelium layer consisting of rough Ck5+ basal cells and Ck18+ luminal cells. It was confirmed that the urothelial cells are mainly surrounded by connective stroma containing vimentin+ stromal fibroblasts and CD31+ endothelial cells, which cover an outer muscle layer mainly consisting of α-smooth muscle actin (α-SMA)+ smooth muscle cells and CD31+ endothelial cells.

From this result, as shown in FIGS. 2H and 9E, it was confirmed that, bladder epithelium, stroma and a muscle layer were successfully reconstituted in vitro such that the three-layered miniature bladder shows a bladder-like tissue structure and cell composition in vivo.

3-4. Confirmation of Hh Pathway Activity Response of In Vitro Reconstituted Three-Layered Miniature Bladder

To further confirm that the three-layered miniature bladders respond to stromal Hh pathway activity, similar experiments as described in Example 3-2 were performed using the reconstituted bladder organoids with stroma.

As a result, as shown in FIGS. 2I, 2J and 10C, it was confirmed that the pharmacological inhibition of the stromal Hh response with Vismodegib in the three-layered miniature bladders decreases the proliferation of epithelial and stromal cells, the upregulation of stromal Hh activity with SAG increases the proliferation of epithelial cells and stromal cells, and based on this, the three-layered miniature bladders repeated the in vivo interaction between the epithelium and stroma to induce cell proliferation.

### Example 4. Confirmation of In Vivo Bladder Physiological Activity of Miniature Bladder During UTI

4-1. Confirmation of Recapitulation of Injury-Induced In Vivo Interaction Between Epithelial Cells and Stromal Cells of Three-Layered Miniature Bladder

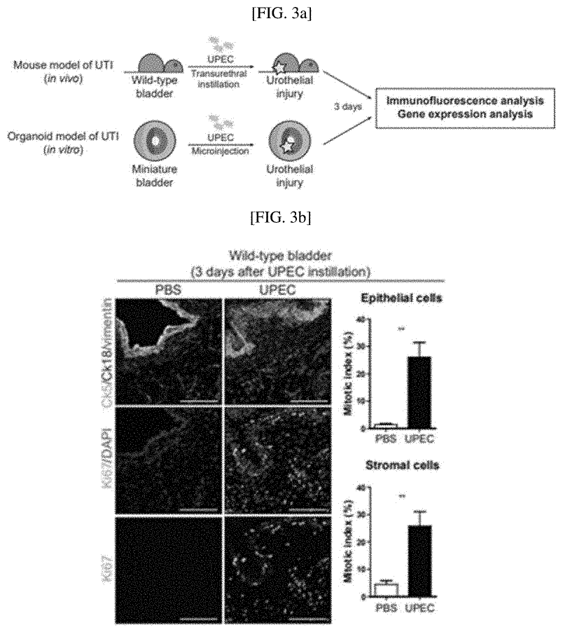

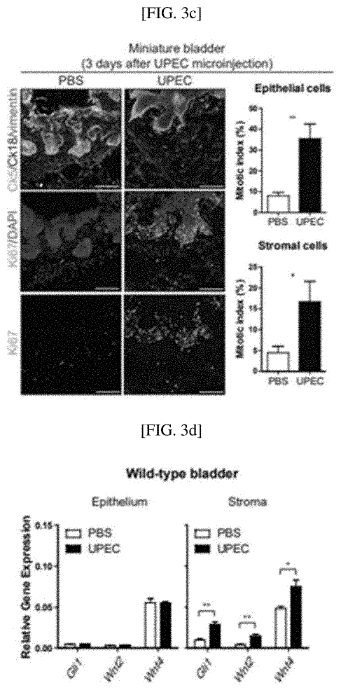

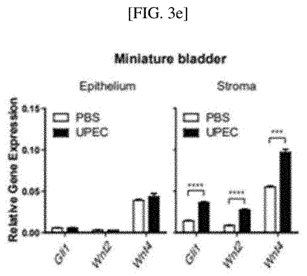

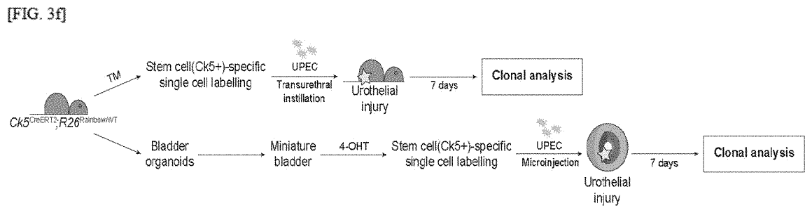

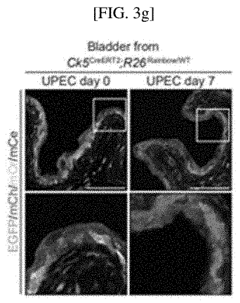

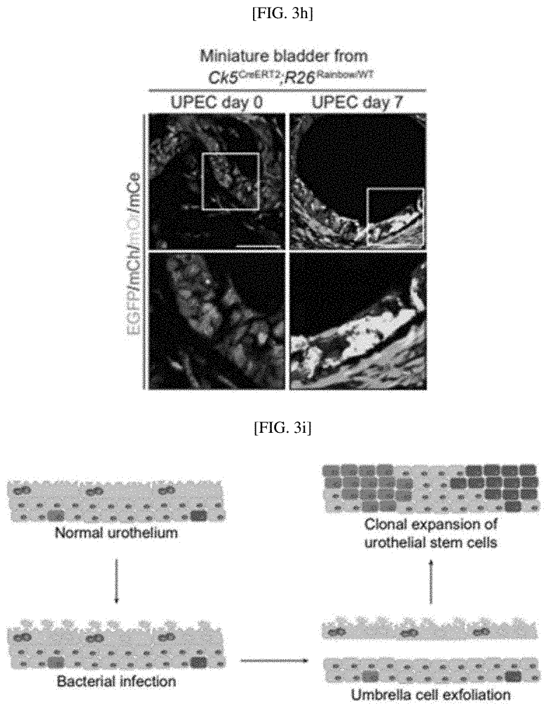

To confirm the possibility of establishing an in vitro UTI model and recapitulating injury-induced the in vivo interaction between epithelial cells and stromal cells in a three-layered miniature bladder, as shown in FIG. 3A, an injury was induced in the miniature bladder by microinjection of UTI89, which is a uropathogenic strain of E coli (UPEC), into the lumen of the miniature bladder, and the regenerative response of the miniature bladder was compared with that of an in vivo mouse model of UTI.

The Ck18+ umbrella cells of a wild-type bladder were exfoliated by bacterial infection, and as shown in FIG. 3B, three days after the bacterial infection, the expression of Ki67, which is a proliferation marker for epithelial cells and stromal cells, significantly increases, indicating injury-induced cell proliferation.

As a result, as shown in FIG. 3C, in an in vitro UTI miniature bladder, UPEC microinjection induces the exfoliation of Ck+18 umbrella cells, and the proliferation of epithelial cells and stromal cells increases, confirming that the cell proliferation response of in vivo bladder is repeated during UTI.

4-2. Confirmation of Regulation of Regenerative Response of Miniature Bladder In Vitro by Injury Induction Between Endothelial Cells and Stromal Cells

To confirm whether injury induction between endothelial cells and stromal cells whose activity are increased by Hh/Wnt signaling feedback regulates the regenerative response of a miniature bladder in vitro, the expression of various genes involved in Hh/Wnt signaling activity was investigated.

As a result, as shown in FIG. 3D, it was confirmed that the stromal Hh response shown by Gil1 expression and stromal expression of Wnt genes including Wnt2 and Wnt4 significantly increase in an injury-dependent manner in an in vivo UTI mouse model.

In addition, as shown in FIG. 3E, in an infected miniature bladder, like an in vivo UTI bladder, the expression of Gil1, Wnt2 and Wnt4 significantly increases only in stroma, rather than epithelial cells, confirming that an in vivo regenerative response with increased activity of a Hh/Wnt signaling pathway in bacterial injury is shown in in vitro UTI with the three-layered miniature bladder.

4-3. Confirmation of Tissue Dynamics of Injury-Induced Urothelial Cell Regeneration by Clonal Analysis Using In Vivo and In Vitro Models of UTI

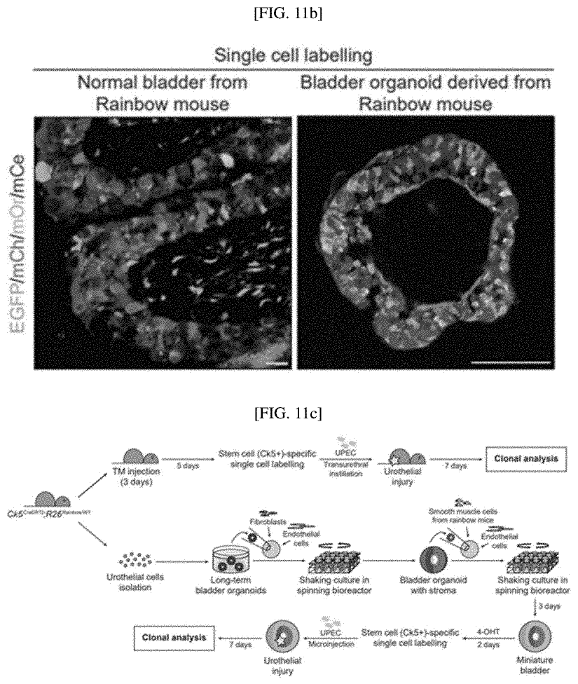

To investigate stem cells and tissue dynamics for urothelial regeneration, as shown in FIG. 11A, first, EGFP was expressed, but to express one of additional three fluorescent proteins in Cre-mediated recombination induced by tamoxifen (TM), “Rainbow mice (R26^{Rainbow/WT}) in which cells are individually recombined were used.

Accordingly, as shown in FIG. 11B, it was confirmed that individual basal cells in TM-treated CK5^{CreERT2}; R26^{Rainbow/Rainbow} mouse bladders are classified in approximately equal numbers of probabilities by persistent and heritable expression of one of four distinct fluorescent proteins.

As shown in FIGS. 3E and 3F, one week after UTI, animal bladders were marked in a single color, and some urothelial cell patches disposed over a wide range of adjacent cells were developed, thereby confirming oligoclonal expansion of urothelial stem cells for reproduction of urothelial cells.

4-4. Confirmation of Reproduction of Clonal Expansion of Basal Cells Regenerating Urothelial Cells in In Vitro UTI Model Developed Using Three-Layered Miniature Bladder

To confirm clonal expansion reproduction of basal cells regenerating urothelial cells in an in vitro UTI model developed using a three-layered miniature bladder, as shown in FIG. 11C, “Rainbow miniature bladder” was manufactured by reconstituting long-term cultured bladder tissue having stroma and an external muscle layer in CK5^{CreERT2}; R26^{Rainbow/Rainbow} mice.

As shown in FIGS. 3F and 11C, to confirm colonality of the expansion of injured urothelial cells caused by UTI using the Rainbow miniature bladder, lineage tracing experiments were performed.

As a result, as shown in FIG. 3H, it was confirmed that clonal patches marked in a single color are generated 7 days after bacterial infection, and as shown in FIG. 3G, as observed in in vivo Rainbow bladders, a regenerated portion of the urothelial cells is clonally generated from the progeny of basal stem cells during injury.

From this result, as shown in FIG. 3I, it was confirmed that, by using in vivo and in vitro models of UTI, a small number of basal stem cells along with the expansion of urothelial cells regenerate the bladder epithelium through oligoclonal expansion in bacterial injury, indicating that the bladder miniature model has a potential as an in vitro disease model system exhibiting in vivo biological characteristics.

### Example 5. Confirmation of Recapitulation of Histopathology of Human Urothelial Carcinoma by Three-Dimensionally Reconstituted Bladder Tumor Organoids Derived from Different Subtypes of Patients

5-1 Immunohistochemical Analysis of Bladder Tumor Organoids

TABLE 1

| | | | | | | Neoad- | |
|---|---|---|---|---|---|---|---|
| | | | | | Intra- | juvant | |
| | | | Tumor stage | Tissue | vesical | chemo- | Recur- |
| # | Sex | Age | and grade | source | therapy | therapy | rence |
| 1 | M | 79 | T2(High) | TURB | N | N/A | Y |
| 2 | M | 58 | T2(High) | TURB | BCG | N/A | Y |
| 3 | M | 68 | T2(High) | TURB | N | N/A | N |
| 4 | F | 56 | T1(High) | TURB | N | N/A | N |
| 5 | M | 72 | T1(High) | TURB | N | N/A | N |
| 6 | M | 81 | T2(High) | TURB | N | N/A | N |
| 7 | M | 59 | T1(High) | TURB | N | N/A | N |
| 8 | F | 73 | T2a(High) | Cystec- | N | N/A | Y |
| | | | | tomy | | | |

As shown in Table 1, in addition to normal bladder organoids, 8 invasive urothelial carcinoma patient-derived bladder tumor tissues were acquired from fresh patient TURB or radical cystectomy samples.

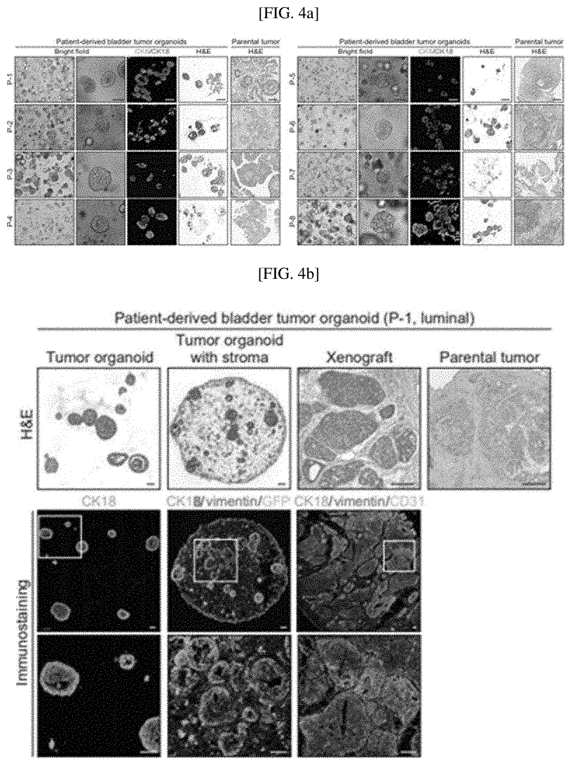

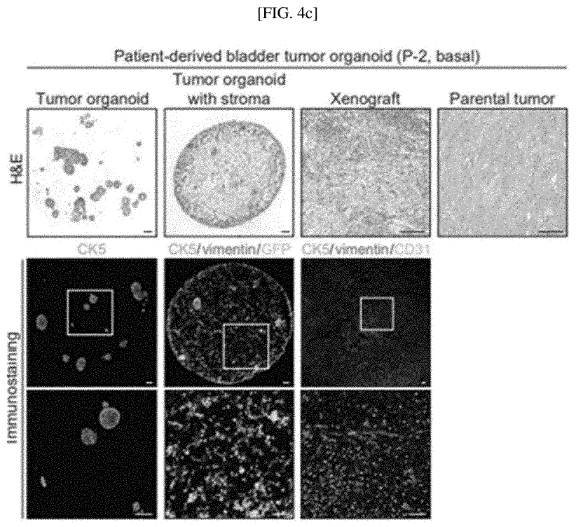

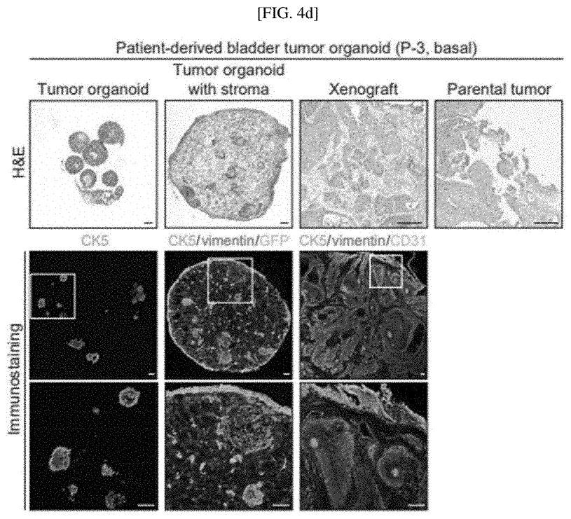

As shown in FIG. 4A, the survival and growth of epithelial tumor cells were promoted by developing optimized culture conditions through normal bladder organoid culture, and eight patient-derived bladder tumor organoids matching eight patients were established.

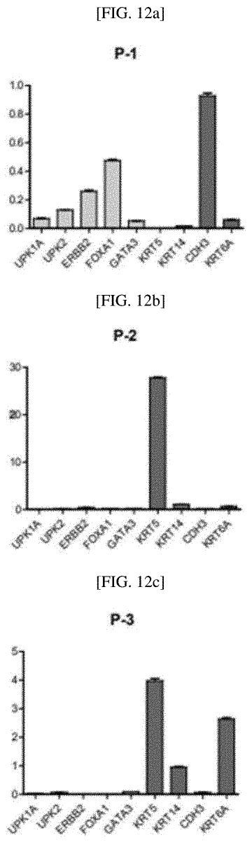

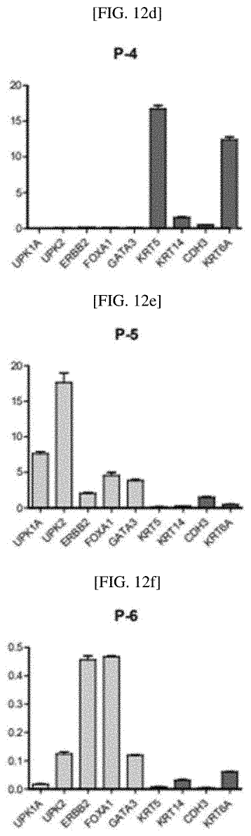

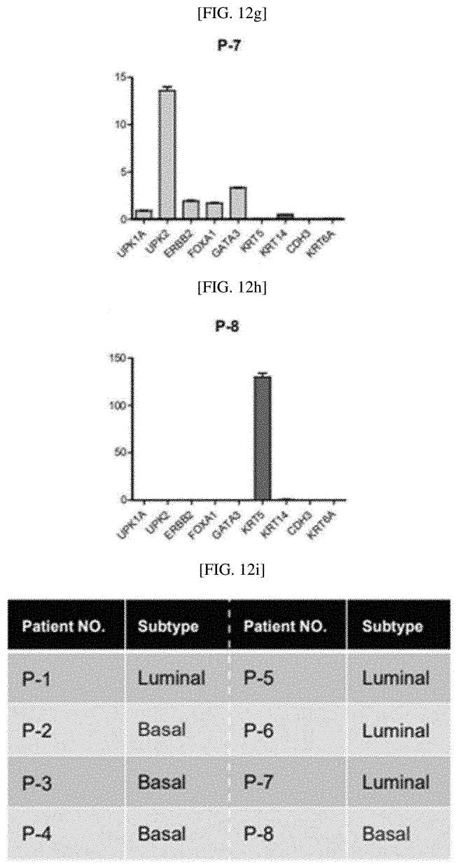

As shown in FIGS. 4A, 12A, 12E, 12F, 12G and 12I, bladder tumor organoid lines derived from four patients (P-1, P-5, P-6 and P-7) showed luminal subtypes with a significant increase in the expression of luminal markers. As shown in FIGS. 4A, 12B, 12C, 12D, 12H and 12I, the bladder tumor organoid lines derived from four patients (P-2, P-3, P-4 and P-8) exhibited basal phenotypes with an increase in the expression of basal markers.

As shown in FIGS. 4A and 12, immunohistochemical analysis revealed that all established basal subtype tumor organoids dominantly expressed Ck5, whereas luminal subtype tumor organoids mainly expressed Ck18, which is the same as subtype analysis RT-PCR results.

In addition, as shown in FIG. 4A, histological analysis of each organ revealed that all bladder organoids generally showed spherical morphologies, and the histopathological characteristics of each organ were concordant with its parental tumor. Particularly, it was confirmed that all tumor organoids not only show cancer cells of a parental tumor, but also do not show the structure of the surrounding tumor stroma of parental tumor tissue.

5-2. Confirmation of Pathophysiology and Mimetic Structure of Parenteral Tumor of Bladder Tumor Organoid

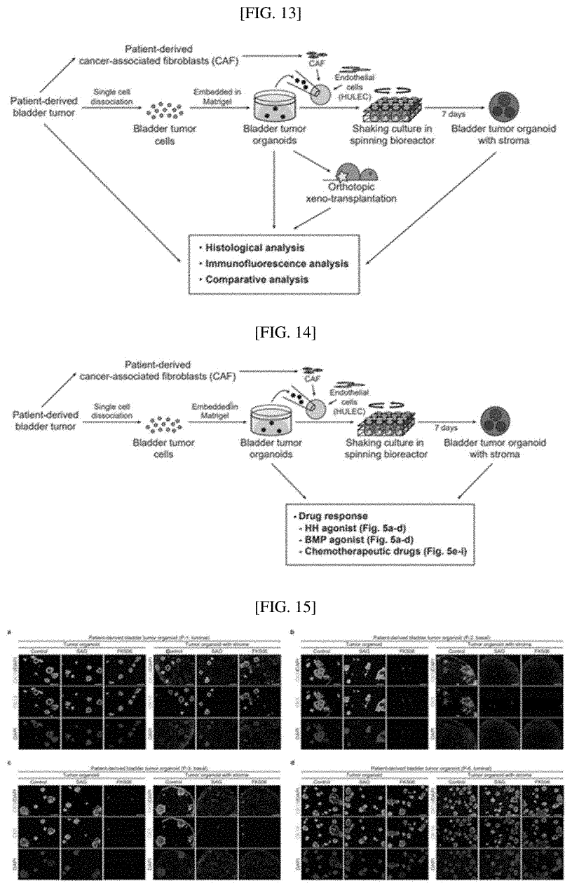

To establish patient-specific tumor organoid models precisely mimicking the pathophysiology and structure of parenteral tumors, as shown in FIG. 13, patient-derived bladder tumor organoids with stroma were produced in vitro by three-dimensionally reconstituting four representative patient-derived bladder tumor organoids including two luminal types and two basal types along with patient-derived tumor-associated fibroblasts (CAFs) and endothelial cells.

A tumor structure in reconstituted bladder tumor organoids was analyzed, and responses of the reconstituted bladder tumor organoids were compared with orthotopically transplanted xenografts and parenteral tumors by H&E assay and immunostaining for Ck5 and Ck18 to mark tumor cells, vimentin for CAFs and CD31 for endothelial cells.

As a result, as shown in FIGS. 4B to 4E, four tumor organoids including dense aggregates of tumor cells with a relatively high nucleus-cytoplasm ratio maintained a spheroidal shape in a tumor tissue culture.

In addition, as shown in FIGS. 4B and 4E, in the case of reconstituted P-1 or P-6 luminal subtype tumor organoids, it was confirmed that CK18+ tumor organoids grew into multiple masses while being isolated by the surrounding stroma including vimentin+ CAFs along with endothelial cells.

In addition, as shown in FIGS. 4B and 4E, it was confirmed that since Ck18+ tumors formed small or large sized nests or clusters with a clear boundary distinct from a stromal compartment, the tumor organoids with stroma precisely mimic the histopathology of xenografts and parenteral tumors.

As shown in FIGS. 4C and 4D, the change in a morphological pattern of tumor growth is more distinctively shown in the basal subtype tumor organoids.

As shown in FIG. 4C, in the case of P-2 basal tumor organoids with stroma, tumor cells showed more migratory characteristics in that CK5+ tumor cells are unevenly distributed in the stroma, and there are indistinct boundaries between tumor cells and stromal cells.

As shown in FIG. 4C, the characteristic of stromal intervention in a xenograft and a parental tumor clearly shows that tumors are usually diffused into stroma containing stromal fibroblasts and infiltrated blood vessels.

As shown in FIG. 4D, unlike P-2 tumors, P-3 tumors showed that tumors with irregular shapes expand the lamina propria in a destructive manner, leading to stromal invasion to show an inverted pattern of tumor stroma, based on a xenograft and the histopathology of parental tumors.

As shown in FIG. 4D, the characteristics of reconstituted P-3 tumor organoids with stroma were confirmed in that tumor cells having spherical morphology in organoid-only culture were partially lost, and a stromal component enclosing the surrounding stroma is cut after CK5+ tumors had grown along the surface of the stromal component in the presence of the inverted patter of tumor stroma.

From this result, it was confirmed that tumor organoids which matched CAFs and three-dimensionally reconstituted from different subtypes of invasive urothelial carcinoma show the intrinsic structure and histopathology of parental tumors, which were not observed in an organoid-only culture condition.

### Example 6. Confirmation of In Vivo Tumor Response of Reconstituted Bladder Tumor Organoid to Stroma-Mediated, Subtype-Dependent Anticancer Agent

6-1. Confirmation of Stromal Hh Activating Effect of Bladder Tumor Organoid

As shown in FIG. 14, an in vitro platform of patient-derived tumor organoids with stroma was established, and then it was confirmed whether, when treated with a Hh or Bmp agonist, bladder tumor organoids exhibit a stromal Hh activity-mediated, cancer-suppressing effect.

To examine the effect of an increased Bmp response in tumor cells, bladder tumor organoids with or without tumor stroma were treated with FK506, known to stimulate a Bmp response.

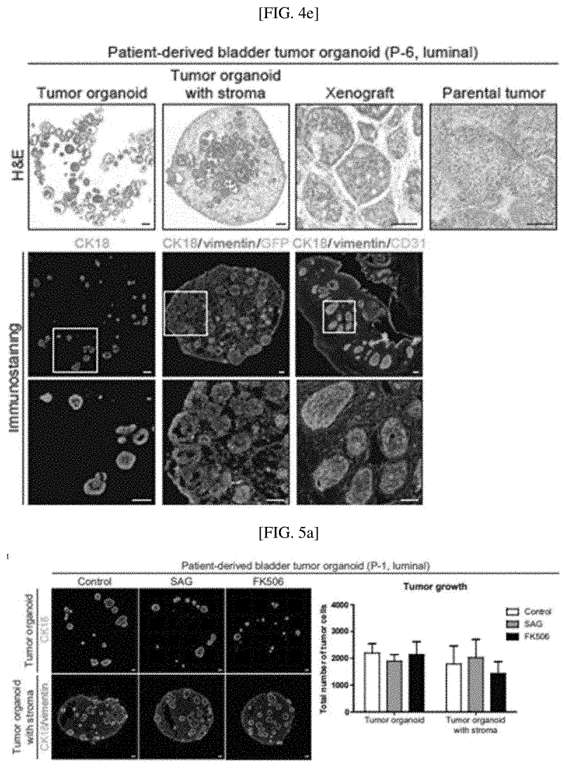

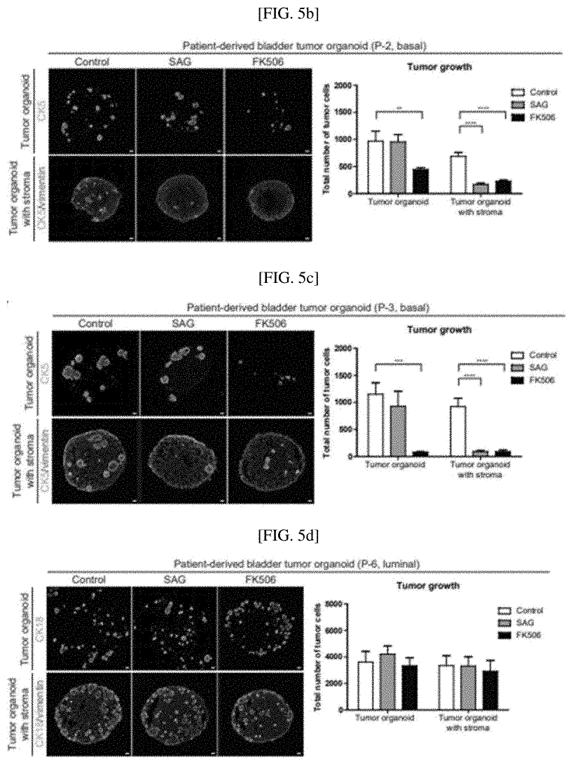

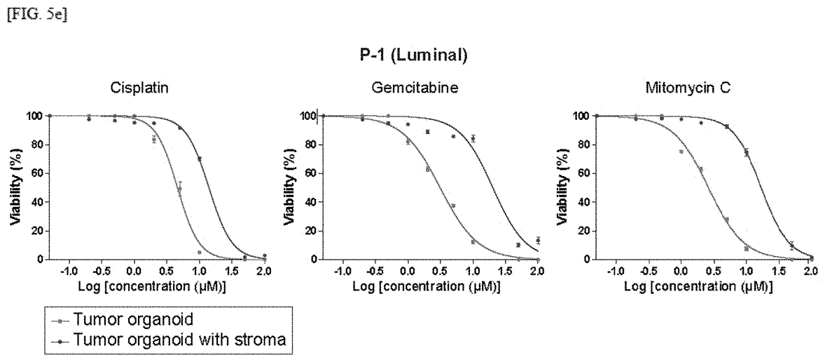

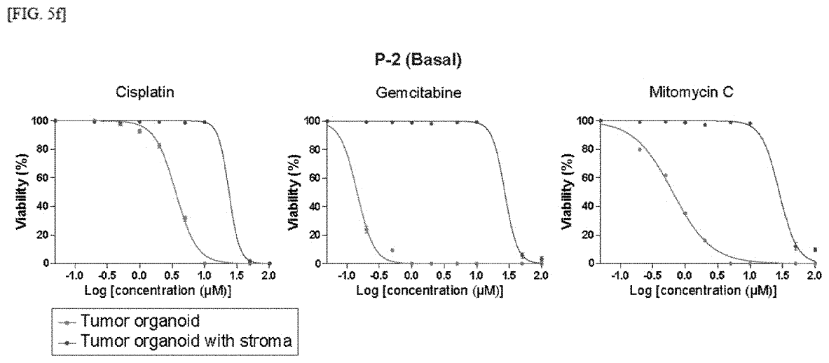

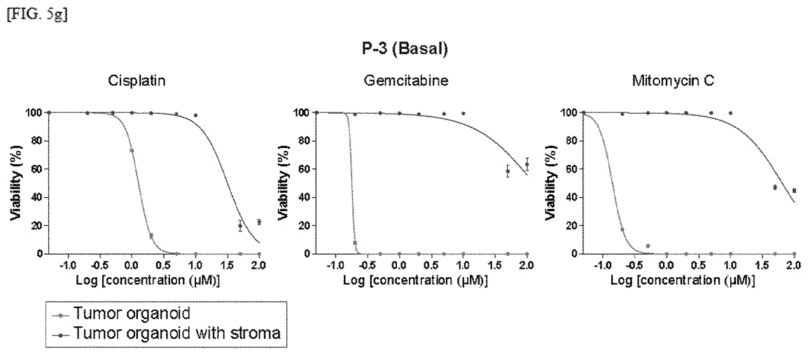

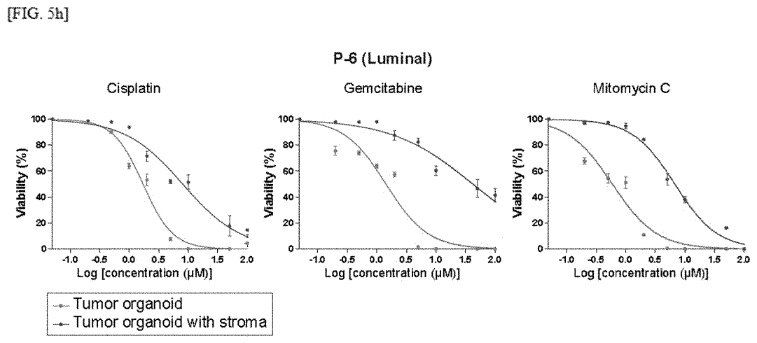

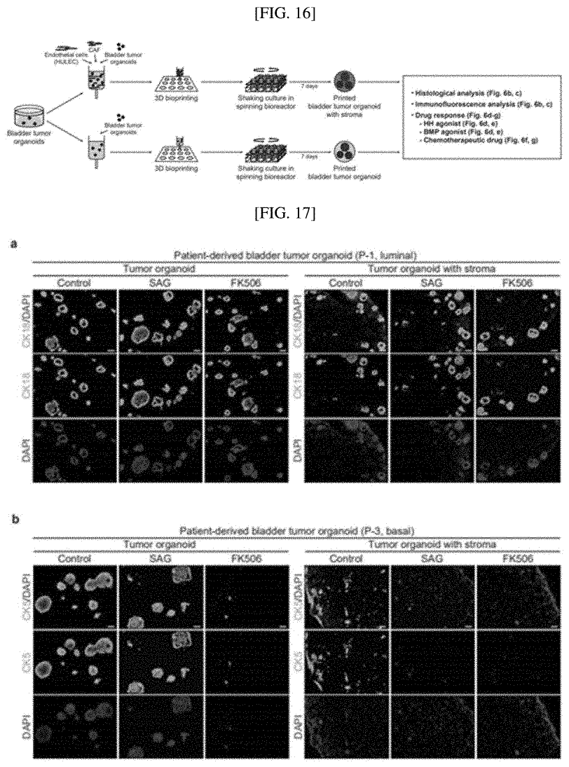

As a result, as shown in FIGS. 5A to 5D and 15A to 15D, the growth of basal tumor organoids (P-2 and P-3) was dramatically reduced regardless of the presence of tumor stroma, whereas luminal tumor organoids (P-1 and P-6) did not respond to the Bmp agonist.

To further examine the effect of stromal Hh activity on bladder tumor growth, bladder tumor organoids were treated with SAG, which is a Hh pathway agonist.

As a result, as shown in FIGS. 5A to 5D, it was confirmed that the growth of only reconstituted tumor organoids of basal subtypes (P-2 and P-3) in the presence of tumor stroma was significantly reduced in response to SAG, and basal subtype tumor organoids without tumor stroma or reconstituted luminal tumor organoids do not respond to a stromal Hh pathway agonist.

This result revealed that reconstituted bladder tumor organoids show an in vivo tumor-like response to a stroma-mediated, subtype-dependent anticancer agent, demonstrating that a stromal Hh response induced stromal expression of Bmp to elicit a tumor-suppressing effect in basal tumors, and a tumor-suppressing effect by subtype conversion from invasive tumors to less aggressive luminal subtypes.

6-2. Confirmation of Chemotherapeutic Response of Tumor Stroma

As shown in FIG. 14, the effects of the tumor responses to conventional chemotherapeutic drugs such as cisplatin, gemcitabine and mitomycin C and the potential of reconstituted bladder tumor organoids with stroma for evaluating drug responses were further confirmed.

As shown in FIGS. 5E to 5I, it was confirmed that, when responses of conventional tumor organoids without stroma were confirmed, luminal tumor organoids are more resistant to all chemotherapeutic drugs than basal tumor organoids.

In addition, as shown in FIGS. 5E to 5I, since each tumor organoid with stroma showed the tendency of shifting a IC₅₀ value to the right in a dose-response curve, compared with bladder tumor organoids without stroma, all reconstituted bladder tumor organoids showed a significant decrease for the three chemotherapeutic drugs regardless of subtypes.

From this result, it was confirmed that chemotherapeutic drugs are poorly delivered to a cancer tissue site due to the surrounding stroma, which is more extensive and denser than normal tissues in many solid tumors, and tumor organoids can precisely exhibit in vivo responses to various chemotherapeutic drugs.

### Example 7. Confirmation that 3D Bioprinting-Based Reconstituted Bladder Tumor Organoid Recapitulates Pathophysiology of Patient-Derived Urothelial Carcinoma

7-1. Confirmation of Histopathology and Tumor Structure of 3D Bioprinting-Based Reconstituted Bladder Tumor Organoid

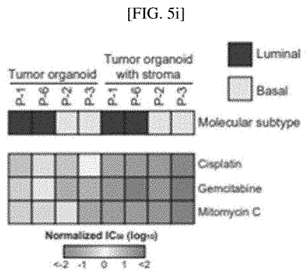

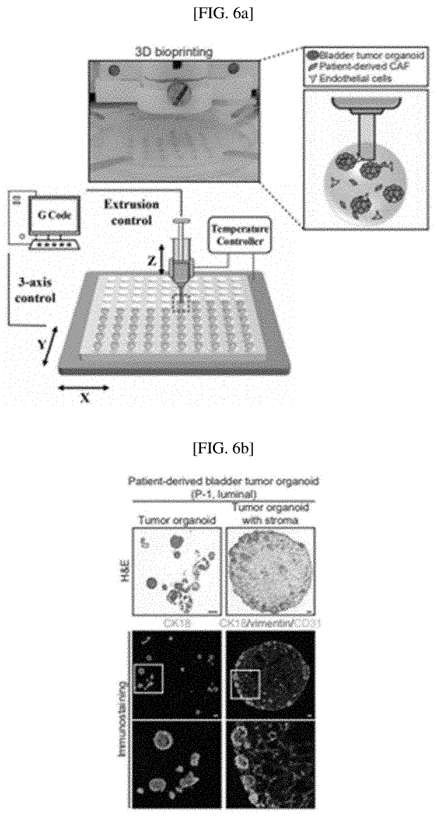

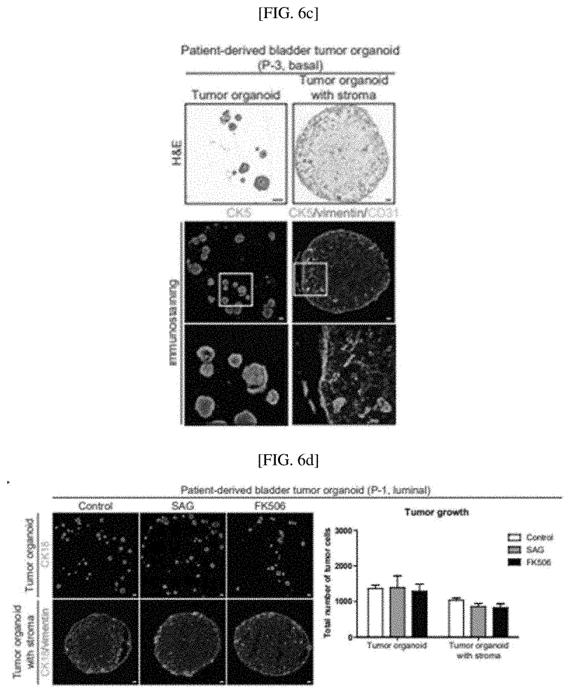

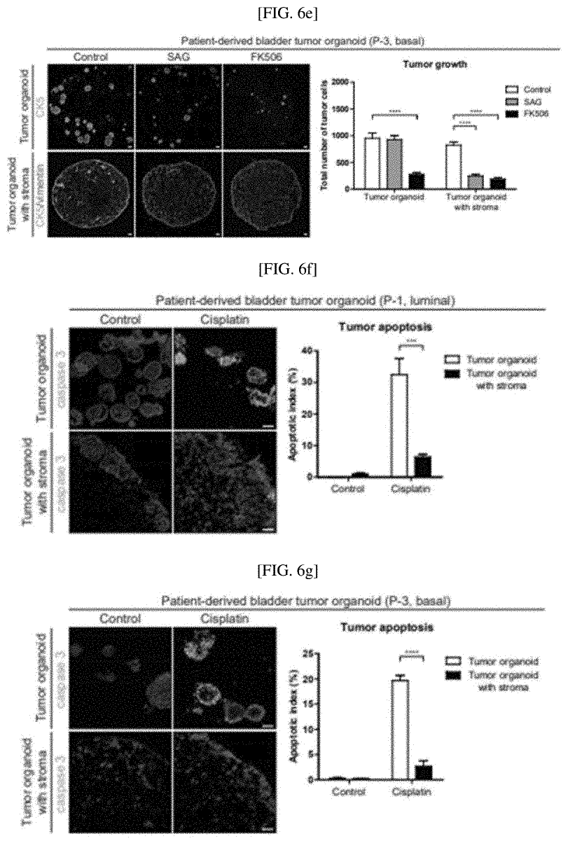

To develop a high-throughput platform for reconstituted tumor organoids, a method of automatically generating multiple reproducible tumor organoids with stroma was designed using a 3D bioprinting technique, and to this end, as shown in FIG. 6A, a screw-driven microextrusion bioprinter enabling controlled patterning of multiple tumor organoids and stromal compartments was developed. A 5×6 array of multiple tumor organoids with stroma, which consists of patient-derived bladder tumor organoids, patient-derived CAFs and endothelial cells, was produced using a bioprinter having a three-axis motorized stage system. As shown in FIG. 16, using the 3D bioprinting platform, patient-derived bladder tumor organoids with stroma were reconstituted from two representative patient-derived bladder tumor organoids lines, including one luminal subtype and one basal subtype. The histopathology and tissue structures of the 3D bioprinting-based, reconstituted tumor organoids were analyzed.

As a result, as shown in FIGS. 6B and 6C, in contrast to tumors in which only the entire rotating oval shaped tumor tissue phenotype was printed (FIGS. 4B and 4D), 3D bioprinting showed that all reconstituted tumor organoids with stroma maintain an intrinsic tumor structure. More specifically, as shown in FIG. 6B, it was confirmed that bioprinted tumor organoids derived from luminal P-1 organoids showed that, according to the development of Ck18+ tumors into several masses isolated by surrounding vimentin+ fibroblasts and CD31+ endothelial cells, tumor nests were generated while being spaced with stroma. In addition, as shown in FIGS. 6C and 4D, in the case of a P-3 organoid-derived bioprinted basal tumor, CK5+ tumors protruded out of a stromal compartment, and the propagation pattern of tumors around stroma in a direction of enclosing the tumor stroma containing fibroblasts and endothelial cells was shown.

7-2. Confirmation of In Vivo Tumor Responses of 3D Bioprinting-Based Reconstituted Bladder Tumor Organoid to Stroma-Mediated Subtype-Dependent Anticancer Agent

In addition to examining histopathology and tumor structure, it was confirmed whether bioprinted bladder tumor organoids exhibit in vivo tumor responses to tumor stroma-mediated, subtype-dependent anticancer agents.

As a result, as shown in FIGS. 6D and 17A, it was confirmed that printed tumor organoids derived from a luminal P-1 tumor does not exhibit a significant change in tumor growth in response to SAG or FK506 regardless of tumor stroma. However, as shown in FIGS. 6E and 17B, it was confirmed that bioprinted basal tumor organoids respond to FK506 regardless of tumor stroma, whereas tumor growth is significantly inhibited only by SAG treatment in the presence of stroma.

In addition, as shown in FIGS. 5E, 5G and 5I, it was confirmed whether bioprinted tumor organoids with stroma show a decrease in response to chemotherapeutic drugs similar to that shown in reconstituted bladder tumor organoids.

As a result, as shown in FIGS. 6F and 6G, it was confirmed that, in luminal and basal tumor-derived printed tumor organoids, tumor cell apoptosis was increased in response to cisplatin without tumor stroma, and such response is significantly reduced when reconstituted with tumor stroma.

From this result, it was confirmed that 3D bioprinting-based reconstitution of patient-derived bladder tumor organoids formed functional tumor organoids that exhibit various characteristics of parental tumors, such as original tumor structures, tumor stroma-mediated anticancer effects and responses to chemotherapeutic drugs.

### Example 8. Confirmation of Tumor Invasion into Muscle Layer and Immune Cell Infiltration of Reconstituted Bladder Tumor Organoid Having Tumor Microenvironment

8-1. Confirmation of Reconstitution of Muscle Layer of Reconstituted Tumor Organoid Platform

A muscle layer was reconstructed to exhibit muscle invasion of tumor cells in an in vivo tumor organoid model. Tumor organoids derived from two different lines, consisting of a P-7 line for the luminal type at the T1 stage and a P-3 line for the basal type at the T2 stage were reconstituted by matching with patient-derived CAFs.

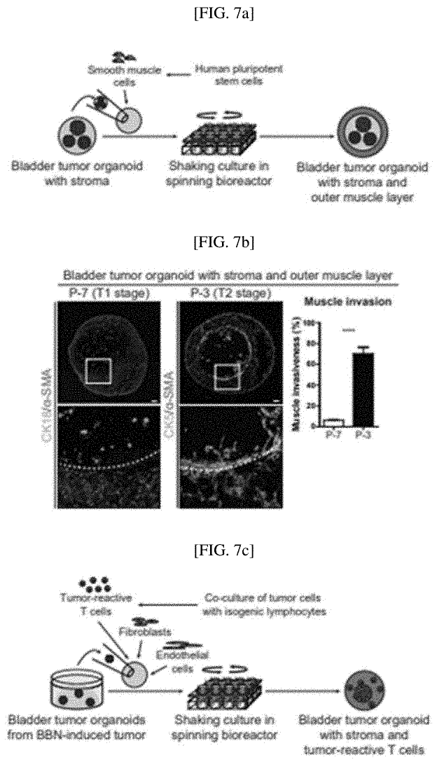

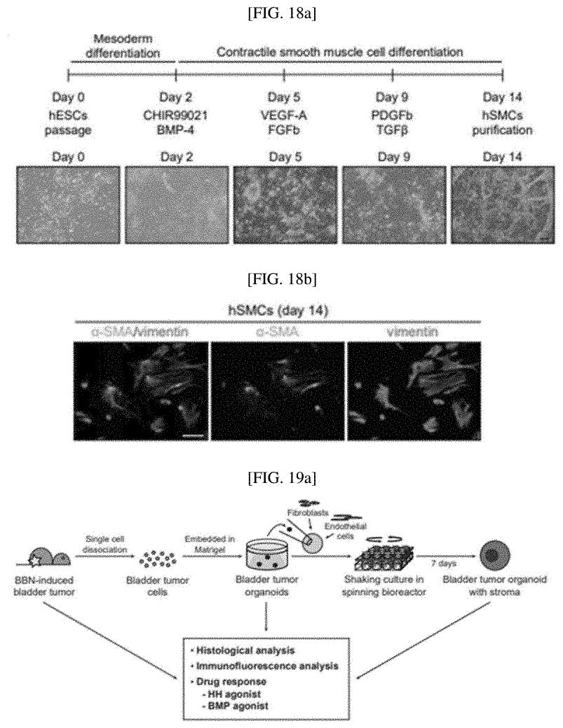

As shown in FIGS. 7A, 18A and 18B, an outer muscle layer was reconstructed with reconstituted bladder tumor organoids using contractile smooth muscle cells differentiated from human PSCs in vivo, and it was confirmed that the multilayered tumor organoids show muscle invasion similar to original tumor tissues.

As a result, as shown in FIG. 7B, it was confirmed that, at the T1 stage, luminal P-7-derived reconstituted tumor organoids propagate in the stromal compartment but did not migrate to the muscle layer, whereas at the T2 stage, tumor organoids derived from the basal P-3 line protrude out of the stromal compartment and 70% of tumor cells migrate to a muscle layer in order to reach the stroma-muscle boundary, compared with 6% of tumor cells in P-7-derived organoids.

8-2. Confirmation of Repopulation of T Cell-Based Immune Microenvironment in Reconstituted Tumor Organoid Platform

To confirm whether a T cell-based immune microenvironment was able to be repopulated in a reconstituted tumor organoid platform, reconstitution of tumor organoids including tumor-specific T cells was performed. To confirm reconstitution of T cells in the tumor organoids, mouse tumor organoids were reconstituted in a bladder cancer mouse model such as an N-butyl-N-4-hydroxybutyl nitrosamine (BBN)-induced mouse model, and comparative analysis was performed.

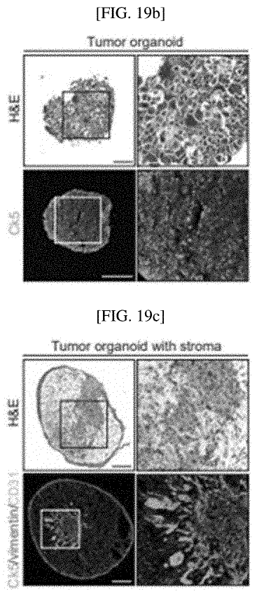

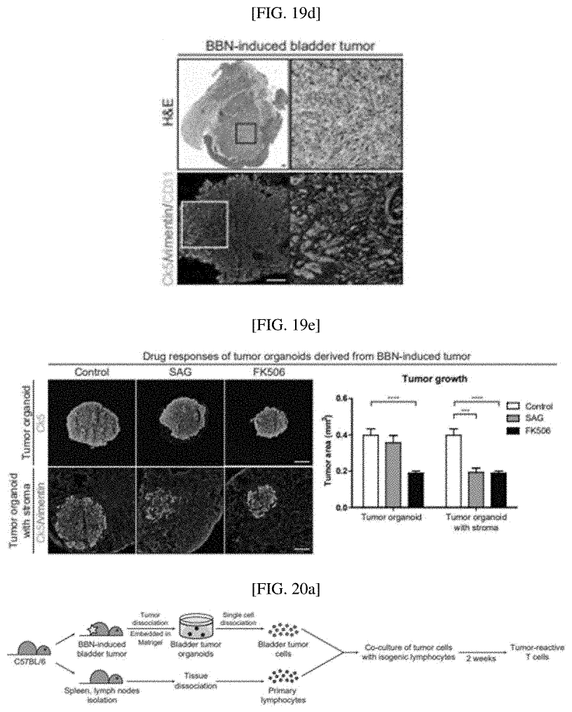

As shown in FIG. 19A, differences between reconstituted mouse bladder tumor organoids and endogenous BBN-induced urothelial carcinoma were confirmed. As in reconstituted tumor organoids, it was confirmed that BBN-induced tumor organoids with stroma show a growth pattern in that tumor cells with nuclear atypia, including polymorphism, which is a representative characteristic of the histopathology of an original BBN-induced bladder tumor, are scattered throughout the stroma.

In addition, as shown in FIG. 19E, it was confirmed that tumor growth is inhibited when the pharmacological activation of the stromal Hh response is performed by treating the reconstituted tumor organoids with stroma with SAG, whereas the growth of tumor organoids is reduced regardless of the presence of stroma when the reconstituted tumor organoids are treated with FK506.

8-3. Confirmation of Reconstitution of Tumor-Reactive T Cells in Mouse Tumor Organoids

In the mouse tumor organoids, to confirm further reconstitution of tumor-reactive T cells, tumor-reactive T cells were prepared by co-culturing urothelial carcinoma-derived mouse tumor cells with isogenic primary lymphocytes in a BBN-induced mouse for 2 weeks.

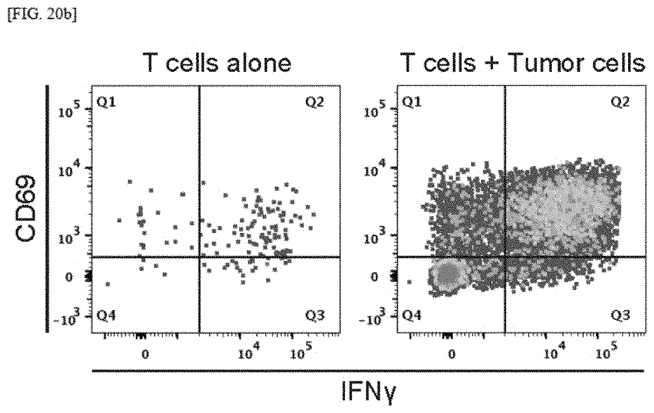

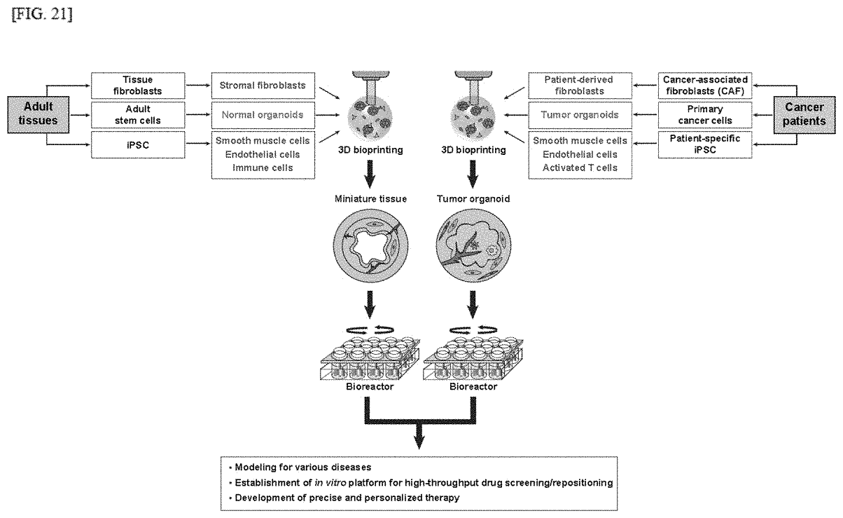

As shown in FIG. 20B, the tumor-reactivity of CD8 T cells was evaluated by flow cytometry analysis, confirming that a tumor-reactive CD8 T cell population was increased approximately 53-fold with respect to IFNγ+ and CD69+ cells after 2 weeks of co-culture.

As a result, as shown in FIG. 7C, it was confirmed that reconstituted mouse tumor organoids having T cell-based immune microenvironments, which include tumor-reactive T cells, stromal fibroblasts and endothelial cells, are generated.

In addition, as shown in FIG. 7D, it was confirmed that, in reconstituted tumor organoids including tumor-reactive T cells, CD8 T cells infiltrate into tumors to induce extensive cell apoptosis, confirming that a tumor mass is remarkably decreased.

From this result, it was confirmed that in vitro organoids with stroma were reconstituted to form a potent platform for modeling various biological aspects of tumors including muscle invasion and immune cell infiltration.

It is confirmed that a stem cell- or tumor cell-based multicellular mimetic tissue structure designed according to the present invention mimics physiological and pathological characteristics of in vivo tissues by realizing the major factors of a tissue microenvironment such as stromal cells, vascular cells, immune cells, and muscle cells in the existing organoid through tissue reconstruction, and the mimetic tissue structure is expected to be effectively used as a platform for new drug development and a disease model by culturing normal tissue and tumor tissue.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

What is claimed is:

1. A method of manufacturing a 3D mimetic tissue structure, comprising the following steps:
(a) preparing an organoid;
(b) culturing the organoid in a medium containing fibroblasts and endothelial cells; and
(c) culturing the organoid cultured in step (b) in a medium containing muscle cells.

2. The method of claim 1, wherein the organoid in step (a) is manufactured by culturing any one of stem cells and tumor tissue.

3. The method of claim 2, wherein the stem cells are urothelial stem cells.

4. The method of claim 1, wherein the tumor tissue is a bladder tumor tissue derived from a patient undergoing transurethral resection of bladder tumor (TURBT) or a cystectomy.

5. The method of claim 1, wherein the fibroblasts are selected from the group consisting of mouse embryonic fibroblasts (MEFs) and cancer-associated fibroblasts (CAFs).

6. The method of claim 1, wherein the muscle cells are selected from the group consisting of primary bladder smooth muscle cells (BSMCs) and human smooth muscle cells (hSMCs).

7. A 3D mimetic tissue structure manufactured according to claim 1.

8. The 3D mimetic tissue structure of claim 7, wherein the tissue is bladder tissue.

9. The 3D mimetic tissue structure of claim 7, wherein the tissue is bladder tumor tissue.
